SEQUENCE LISTING (1 sequence)

SEQ ID NO: 1 - RNA, 2431 nt, Xenopus laevis
  source 1..2431: mol_type = mRNA
cttgactgcagggtttatatagtgctgctaaatttagtctccactgagtgctcagcctcctcatcctgaacgtgtttcctcctctccttgcaattggtttggtgcatgccccccgtgcctttatttataaatctagggcccctctctgcagcaaggaaaacttgtttttgctattgagcgtaacttgatgcacggacgtctccgctacggggatcggggagttcgtcatcctatttaattctctgtggttttttttaattgtgacttttattcccgttttgctgacaatctgattcggatttgggatggctctgagcgagaccattctcccttctttcgctacgtttgcaagccaagataggtggaagtgtgagtatgagtccaagtctccccgcggcagctgtggcattccagatgtaatgcaaagcccagaccccttctcttctaggaaggaagacgaagatttaaacaatgttctggaatttgtgttggccatgggctgtgacaaaagtctccaaacctgtggagacttttcaatggcaccttccgagtcctgcacttactaccaaacaagcccttctcagggaagctacagcacagaccaaagccctcctccttatagttcgggtagcctcatgactgaacttattaggtctgacatggacaactactgccatcctagcaatgttcaagggagattctttgtgtcatcaaattttgactttccagatacaatcaaagcagaacctgacatggattcatatggaccagttatgggtatggttccccagtcctgcacaaagatcaagcaagagggcagtactgcttgtatgatggcatatgaacagcctagacttgctaattctcctcagatgggtggccctgacacccctcctttgagccccgatgacataatgttaagtgaatgccagtcccagttgatgtgccacccttcactgccttatgctcctaattatccgcatgctggcttccatcaccagtcacctatccagtatcaaaatacctcccagtatggcatgtttgaggacaatatgcccatgcaaccttctattgccaggggcatgataacacctccctgctcccctttggaaatattggaaaccaagccaaagagaggccgcagatcttggcctaggaaaaggacggcgtcacacacttgctcttatgctggttgtgggaagacctacacaaagagttcccacttgaaagcccatctcagaacccacacaggtgagaagccttatcactgcaactgggaaggatgtggctggaaatttgcccgatctgatgaactaacgcgccatttccgtaagcatactggccaccggcccttccagtgccatctgtgtgaccgtgccttctctcgatctgaccaccttgcactgcacatgaagaggcacatgtagaactgagacttgtgtgtaaagcaatggaggggcattgggtattctggaacttttcatggccaaatgaactctgccactggctcttcttggagggccatactgtagctggtactgcatggggtggcatttgtccttttgcaactttgctggaccccacgaggaatgacttgactcaaaggctggacctgtatcttgcattaactggaacttttttgagaatgtttgctgggatatgtctgtatgttcatatagtacttaatgattaacactttgccttcttagaaagtggcaaaacgcattttataagggcattgatgagagtaaatctctaacagtagtagtaatgcagagtatctccatacaccttcacctccatttctaaccctttacctaaagtacttaaatgaatgccctggtttaagtccaggtttaagcttcaaaacctgcacttctcccctctttcatttcaccactggaatcaaggtagtgttggtggtgatactctgcattggtacgtgctgcagcctatgtgcatttttctacatgctattgttgattgtaactgcctgtttcagaagacctaggacaatgtttgtatgaaactgccttaattttgttcacgttgacgggatacacgtttgaatatctcataacatttctttttttttttaagtatatgaaattgcataaaggaaaacactcctactcttcaaggaatatatttttatataaacggtgtgttttctcacattacactataaaacagcagggctagaggtcttgcaagagaatagcctttttatatattatatatttatgtaattatatatttgtatatagatttgtaaaattaatgtggtgtatagtgtaaatacgacaactgcaaatggcaactttttttttttatatataatattgggaggggggcatttcttccgtgacatggattgttttttttcttttttattaaagaaaaatataaacaacaaaaaaaaaaaaaaaaa

(19) United States
(12) Patent Application Publication
(10) Pub. No.: US 2025/0333693 A1
(43) Pub. Date: Oct. 30, 2025

(54) CELL TYPE CONVERSION

(71) Applicant: Cambridge Enterprise Limited, Cambridge (GB)

(72) Inventors: Hector BARBOSA TRIANA, Cambridge (GB); John Bertrand GURDON, Cambridge (GB); Ming-Hsuan WEN, Cambridge (GB)

(21) Appl. No.: 19/205,898

(22) Filed: May 12, 2025

Related U.S. Application Data
(63) Continuation of application No. PCT/GB2023/053004, filed on Nov. 15, 2023.

(30) Foreign Application Priority Data
Nov. 15, 2022 (GB) 2217053.4

(51) Int. Cl. C12N 5/0793

(52) U.S. Cl. CPC C12N 5/0619; C12N 2501/40; C12N 2506/02

(57) ABSTRACT

This disclosure relates to a method of somatic cell nuclear reprogramming to alter the cell type comprising preparing a GV extract, permeabilising somatic cells, incubating the somatic cells with the GV extract to alter the cell type, and rescaling somatic cell membranes, wherein said GV extract does not comprise oocyte cytoplasm. The disclosure also relates to a GV extract comprising reprogramming factors.

### REFERENCE TO AN ELECTRONIC SEQUENCE LISTING

The contents of the electronic sequence listing (401C1_SeqListing.xml; Size: 4,277 bytes; and Date of Creation: Nov. 14, 2023) is herein incorporated by reference in its entirety.

### FIELD OF INVENTION

The present invention relates to altering the cell type of somatic cells. More particularly, the present invention relates to altering the cell type of somatic cells using germinal vesicle (GV) extracts.

### INTRODUCTION

The stability of cell differentiation is a requirement to avoid malfunction as cells and tissues get older. We describe here a procedure by which the differentiated status of cells can be reversed, so that pluripotency gene expression is regained by cells that have undergone differentiation, and new unrelated cell types are formed. To achieve this, cells of differentiated tissues are exposed to abundant extracts of amphibian meiotic oocytes. Such reprogrammed cells can be induced to follow new directions of differentiation and so to serve as replacements for malfunctioning adult cells of humans.

The discovery that the four Yamanaka factors when overexpressed in MEFs⁵³ (mouse embryonic fibroblasts) can generate ESC-like cells (induced pluripotent stem cells, iPSCs) has opened the way to derive some kinds of cell-types from adult cells as a source of replacement cells in humans. In the ten years since their result, many labs have tried to determine the route by which this transition can take place³⁻⁵. There are three reasons why this has had only limited success so far. These are (i) the process is slow, usually requiring about 1-3 weeks for pluripotent cells to appear, (ii) the yield of pluripotent cells is small, i.e., only a few of the treated cells being ancestral to the derived pluripotent cells, and (iii) DNA replication and cell division of the treated cells continue during the intervening time and may be necessary for the reprogramming to take place.

We describe here a way of avoiding these limitations and hence towards identification of the earliest steps in this reprogramming process. This involves the use of oocytes (in first meiotic prophase) as opposed to eggs (in second meiotic metaphase) as recipients for transplanted mammalian somatic nuclei². By comparison with previous attempts to unravel the earliest stages of iPSC reprogramming, oocyte nuclear transfer completely avoids the uncertain contribution of cell division and DNA synthesis^{6, 7} and the long reprogramming time⁸ (3-5 days for the earliest markers of iPSC derivation, Thy1 down and SSEA1 up) is greatly shortened to 24 hours at 18° C. Specifically, the method involves the use of GV extract in the absence of oocyte cytoplasm. As such, factors present in the oocyte cytoplasm which may be undesirable for reprogramming are advantageously not present during downstream applications. Furthermore, the method achieves efficient reprogramming in the absence of additional factors, such as protease inhibitors. The GV extract can be optionally modified to express or overexpress factors which have the effect of further increasing the efficiency of reprogramming somatic cells. In addition or alternatively, recombinant factors can be added during the incubation step which also have the effect of further increasing the efficiency of reprogramming somatic cells.

The requirement of cell division for nuclear reprogramming has been unclear for a long time⁹. On the one hand, somatic cell nuclear transfer to metaphase eggs and interphase zygotes shows the former is important for nuclear reprogramming to pluripotency due to its mitotic activity^{10, 11}. On the other hand, the fusion of somatic cells with embryonic stem cells shows the early reprogramming, indicated by the activation of Oct4, is independent of DNA replication and cell division but the later completion of reprogramming may rely on it^{12, 13}. The induction of pluripotency by Yamanaka factors is accompanied by several rounds of cell division for weeks and only a small population of cells become induced pluripotent stem cells^{1, 5}. The induction of pluripotency by Yamanaka factors (OSK), combined with the incubation of MEF in metaphase and interphase egg extracts, shows that nuclear reprogramming requires mitosis to remodel nuclei¹⁴ Here we evaluated the completion of nuclear reprogramming by nuclear transfer to meiotic oocytes which are inactive in cell division and developed the procedure to reprogram human cells to a totipotency-like state for the potential use of cell therapy.

### SUMMARY OF INVENTION

According to a first aspect of the invention, there is provided a method of somatic cell nuclear reprogramming to alter the cell type comprising preparing a GV extract, permeabilising somatic cells, incubating the somatic cells with the GV extract to alter the cell type, and resealing somatic cell membranes, wherein said GV extract does not comprise oocyte cytoplasm.

In one embodiment, the altering the cell type may comprise reprogramming the somatic cell to a progenitor cell or a stem cell.

In one embodiment, the progenitor cell or the stem cell include a unipotent stem cell, oligopotent stem cell, multipotent stem cell, pluripotent stem cell or totipotent stem cell.

In one embodiment, the method may further comprise the differentiation of the progenitor cell or the stem cell to alter the cell type.

In one embodiment the differentiation of the progenitor cell or the stem cell may produce a somatic cell or progenitor thereof, wherein the somatic cell or progenitor thereof is of a different lineage. In one embodiment, the differentiation may comprise the incubation of the progenitor cell or stem cell with differentiation medium. In one embodiment, the differentiation may comprise the addition of one or more differentiation factors.

In one embodiment, the GV extract may comprise one or more reprogramming factors. In one embodiment, the reprogramming factors may be factors which change chromatin accessibility.

In one embodiment, the reprogramming factors may be DNA modifying enzymes, histone variants, histone-modifying enzymes, chromatin remodelers, chromatin modifiers and transcription factors. In one embodiment, the DNA modifying enzymes may be AID or Mbd3. In one embodiment, the histone-modifying enzymes may be kdm4 or kdm6. In one embodiment, the chromatin remodelers may be Brg1. In one embodiment, the transcription factors are Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof.

In one embodiment, the isolated GV may be derived from a meiotic oocyte.

In one embodiment, the GV may be isolated from a Xenopus or mammal or a ray-finned fish. In one embodiment, the mammal may be a human, mouse, porcine or cattle. In one embodiment, the ray-finned fish may be a salmon or a sturgeon.

In one embodiment, the somatic cells may be mammalian cells. In one embodiment, the mammalian cells are human cells, mouse cells, cattle cells, porcine cells, dog cells, cat cells or horse cells.

In one embodiment, the altering the cell type may further comprise the addition of one or more exogenous reprogramming factors.

In one embodiment, the somatic cells and the GV extract, and optionally one or more exogenous reprogramming factors, may be incubated for any one of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours. In one embodiment, the somatic cells and the GV extract, and optionally one or more exogenous reprogramming factors, may be incubated for any one of about 1, 2, 3, 4, 5, 6, 7, 8, 9, or 10 days.

In one embodiment, the progenitor cell or stem cell may be incubated for any one of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days in differentiation medium or 1.5, 2, 3, 4, 5, 6, 7, 8, 9, 10, 11, 12 months in differentiation medium.

In one embodiment, the somatic cells may comprise a selectable or reportable marker, comprising totipotency genes, comprising one or more of Ne1fa, Top2a, Gata2, Eif3h, Dppa2/4, and Atr, pluripotency genes, comprising one or more of Jun, Sox2, Myc, Klf4, Mycn, Klf2, Pou5f1, Utf1, and Sall4, comprising trophoblast genes comprising one or more of Tfap2c, Hand1, Msx2, Csf1r, and Gcm1, and comprising gene markers for somatic cells, comprising Neun, Tubb3, Myod1, Myog, and Itga7.

In one embodiment, the somatic cells may be permeabilised using Streptolysin O, digitonin, lysolecithin, or mixtures thereof.

In one embodiment, the cell membranes may be resealed using CaCl₂.

In one embodiment, the step of preparing a GV extract may comprise mechanical dissociation.

In one embodiment, the method may further comprise a step of modifying the GV extract to express a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof. In one embodiment, the GV extract overexpresses a gene encoding a protein selected from the list comprising Gli1, FoxA, Gata4, Ascl1, Brn2, Myt1l, PU.1, xklf2, xsox2, xklf2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof. In one embodiment, wherein the step of modifying the GV extract comprises injecting the cytoplasm with mRNA or incubating the oocyte with a rare-cutting endonuclease, for example a TALEN, ZFN or CRISPR/Cas9 and guide RNA, optionally, wherein the mRNA encodes a protein selected from the list comprising Gli1, FoxA, Gata4, Ascl1, Brn2, Myt1l, PU.1, xklf2, xsox2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof or the guide RNA comprises a gene encoding Gli1, FoxA, Gata4, Ascl1, Brn2, Myt1l, PU.1, xklf2, xsox2, xklf2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof.

According to a second aspect of the invention, there is provided a GV extract comprising reprogramming factors, optionally, wherein the reprogramming factors are factors which change chromatin accessibility.

In one embodiment, the reprogramming factors may be DNA modifying enzymes, histone variants, histone-modifying enzymes, chromatin remodelers, chromatin modifiers and transcription factors. In one embodiment, the DNA modifying enzymes may be AID or Mbd3. In one embodiment, the histone-modifying enzymes may be kdm4 or kdm6. In one embodiment, the chromatin remodelers may be Brg1. In one embodiment, the transcription factors are Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof.

In one embodiment, the GV may be isolated from a Xenopus or a mammal or a ray-finned fish. In one embodiment, the mammal is a human, mouse, porcine, or cattle. In one embodiment, the ray-finned fish may be a salmon or a sturgeon.

In one embodiment, the GV extract is modified, optionally wherein the GV is modified to express a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, or homologues thereof.

In one embodiment, the GV is modified to overexpress a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, or homologues thereof.

According to a third aspect of the invention, there is provided a cell derived from the method according to the invention.

According to a fourth aspect of the invention, there is provided a cell with an altered cell type derived from the method according to the invention.

According to a fifth aspect of the invention, there is provided a kit comprising a permeabilization agent, a GV extract and/or one or more reprogramming factors, and optionally instructions for use. In one embodiment, the kit may further comprise differentiation medium and/or one or more differentiation factors. In one embodiment, the GV extract is modified, optionally wherein the GV is modified to express or overexpress a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, or homologues thereof.

### FIGURES

The invention is further described in the following non-limiting figures.

FIGS. 1A-1F: The chromatin changes and nuclear envelope breakdown in transplanted MEFs injected in GVs of non-dividing oocytes.

FIG. 1A: Schematic diagram shows the procedures of nuclear transfer to oocytes and following analyses.

FIG. 1B: Heatmap shows similar transcriptional changes for oocyte-induced genes in donor cell types and nuclear transplants at 48 hours after nuclear transfer. Pluripotency genes are indicated at the right side of the heatmap.

FIG. 1C: Images show the dispersion of chromatin (in green) and loss of HP1α (in magenta) in MEF after nuclear transfer. Scale bars indicate 10 μm.

FIG. 1D: Bar chart shows that the chromatin in MEFs dispersed by 2-fold in area at 24 hours after nuclear transfer. Error bars represent±SD. *p<0.05 by the Student's t test, n=3.

FIG. 1E: Bar chart shows the loss of HP1α in three density levels of chromatin in MEFs from 4 hours onwards. At all three time points, the more condensed the chromatin is, the more areas of chromatin occupied by HP1a. Error bars represent±SD. *p<0.05, **p<0.01 by the Student's t test, n=3.

FIG. 1F: Images show the breakdown of nuclear envelope (in magenta) and burst of chromatin (in green) in nuclei of injected MEF after nuclear transfer. The breakdown of nuclear envelope where chromatin burst was observed (white arrowhead) at 4 hours after nuclear transfer. Scale bars indicate 10 μm.

FIGS. 2A-2F: Completion of transcriptional reprogramming to a totipotency-like state by oocytes within 24 hours after nuclear transfer

FIG. 2A and FIG. 2B: Pluripotency genes (FIG. 2A) and trophoblast genes (FIG. 2B) in transplanted MEFs were activated by oocyte factors at Day 1 after nuclear transfer and remain at the same expression level on Day 2.

FIG. 2C: Sox2 transcripts (white spots) were activated by oocyte factors in all transplanted MEFs at 24 hours after nuclear transfer. The red colour shows TO-PRO-1 stain for chromatin of MEFs. The areas outlined in blue show the absence of hybridized probes to the background areas between MEF nuclei.

FIG. 2D: Venn diagram shows similar gene induction by oocyte factors for three cell types at 48 hours after nuclear transfer.

FIG. 2E and FIG. 2F: Bar charts show the different responses between cell types to the induction of pluripotency genes (FIG. 2E) and repression of myogenic genes (FIG. 2F) by oocyte factors at 48 hours after nuclear transfer.

FIGS. 3A-3E: Retention of gene resistance to oocyte reprogramming in differentiated MYO.

FIG. 3A: Venn diagram shows the differentiated MYO nuclei are more resistant to oocyte reprogramming than embryonic ESC and MEF. Among oocyte-inducible genes, the differentially expressed genes (Fold change≥4) indicates the gene resistance in certain cell types with lower gene expression after nuclear transfer. Considering the transcriptional activity of donor cells may affect the results, genes that are down-regulated by oocytes and shown in the oocyte-resistant genes are excluded.

FIG. 3B: Heatmap shows three cell types respond to oocyte reprogramming similarly for the top 1500 highly expressed oocyte-induced genes in MYO-NT. The top 1500 highly expressed genes in MYO-NT includes pluripotency genes, Jun, Klf4 and Myc.

FIG. 3C: Boxplot shows the transcriptional increase in numbers (TPM) for the top 1500 highly expressed oocyte-induced genes in MYO-NT.

FIG. 3D: Heatmap shows the different response among three cell types to oocyte reprogramming for the oocyte-resistant genes in MYO-NT. Oocyte-resistant genes in MYO-NT include six pluripotency genes, Klf2, Sox2, Pou5f1, Sall4, Utf1 and Mycn.

FIG. 3E: Boxplot shows the transcriptional changes in numbers (TPM) for the resistant genes in MYO-NT.

FIGS. 4A-4H: Potential application to humans.

FIG. 4A: Bar chart shows the induction of totipotency genes in mouse cells (ESC, MEF, and MYO) and human cells (hNEU) on Day 2 after nuclear transfer. ESC, n=3; MEF, n=4; MYO, n=3; hNEU, n=3.

FIG. 4B: Bar chart shows the activation of pluripotency genes in human lung stem cells after nuclear transfer.

FIG. 4C: Bar chart shows the increase in gene expression of the neuronal differentiation markers from reprogrammed hLB by GV extracts after neuronal differentiation. GV, treatment of GV extracts; ND, treatment of neuronal differentiation medium.

FIG. 4D: hDF formed and maintained as embryoid bodies at Day 15 after GV extracts treatment. Dark area in the lower figure is an embryoid body.

FIG. 4E: GV-extract treated hDF show the expression of neuronal differentiation markers at Day 21 after neuronal differentiation. NEUN (in green) and TUBB3 (in red) are markers for neuronal differentiation. The nuclei are stained by DAPI (in blue). Scale bar indicates 50 μm.

FIG. 4F: Image shows the unfertilized egg 8 hours after injection of adult human lung stem cell nuclei.

FIG. 4G: Image shows the oocyte in meiotic prophase at 3 days after injection of adult human lung stem cell nuclei.

FIG. 4H: Bar chart shows the activation of pluripotency genes to similar levels in mouse cells (ESC and MEF) and human cells (hNB) on Day 2 after nuclear transfer.

FIGS. 5A-5C: Quantification of chromatin changes shows the dispersion/decondensation of chromatin, also seen in ESC and MEF.

FIG. 5A: Image shows the quantification for chromatin area per cell. Yellow lines circle the areas of chromatin (green) for each cell.

FIG. 5B: Image shows the quantification for areas of chromatin classified to three density levels and the quantification for areas of HP1α. One of injected MEFs in GV of oocyte was magnified to show the details of the classification of chromatin to three density levels. Areas of dense, middle and loose chromatin was circled by yellow lines, between yellow and orange lines, and between orange and purple lines, respectively. Areas of HP1α, overlaid with chromatin of three density levels, was circled by magenta lines.

FIG. 5C: Images show dispersion of chromatin and loss of HP1α in transplanted ESC and MYO after nuclear transfer. The same experimental procedures were applied as in FIG. 1B. Scale bars indicate 10 μm.

FIGS. 6A-6B: Global gene expression shows the induction of a totipotency-like state in MEF by Xenopus oocytes is achieved in 24 hours after nuclear transfer.

FIG. 6A: Heatmap shows that Xenopus oocytes reprogram the transcriptional patterns of donor MEF within 24 hours after nuclear transfer (columns 1-3 versus 4-7). For the expression patterns of donor MEF cells, we showed that the expression patters of MEF are almost identical with or without BrUTP pulldown (column 1 versus 2); the expression patterns of the MEF cell line highly resembles the reference transcriptome of primary MEF in Expression Atlas (column 2 versus 3).

FIG. 6B: Scatter plot shows that the expression patterns of 21,683 oocyte-induced genes in MEF nuclei are almost identical at 24 and 48 hours after nuclear transfer (r=0.78, Pearson correlation coefficient). Within 24 hours, pluripotency genes (black circle, o) and trophoblast genes (black cross, x) are induced by oocytes and remain the same expression level onwards. From 24 to 48 hours after nuclear transfer, 18,706 genes (86% of 21,683 genes) remain the same expression (4>Fold change>0.25, orange dots); 1,390 genes are up-regulated (Fold change≥4, red dots) and 1,587 genes are down-regulated (Fold change≤0.25, green dots).

FIG. 7: Redirection of cell differentiation. Bar chart shows the differentiation-related functions enriched after the overexpression of xklf2 in the GV extract in MYO at 48 hours after nuclear transfer (Gene ontology, adjusted p-value<0.05, n=3).

FIGS. 8A-8F: Enhanced oocyte reprogramming by xklf2 overexpression. Injection of in vitro transcribed xklf2-HA mRNA (SEQ ID NO: 1) 9.2 ug into the cytosol of oocytes 24 hours before nuclear transfer of mammalian nuclei into Xenopus oocytes enhanced the oocyte reprogramming in transplanted cells. In ESC (FIG. 8A and FIG. 8B) and MEF (FIG. 8C and FIG. 8D), the overexpression of xklf2 regulates the pluripotency gene expression. Klf2 and Myc were upregulated and Jun was downregulated in ESC (b, p<0.05, n=3); Klf2, Sall4, and Mycn were upregulated, and Pou5f1 was activated in MEF (d, p<0.05, n=4). In MYO (FIG. 8E and FIG. 8F), the pluripotency genes resistant to oocyte reprogramming were activated by the overexpression of xklf2, including Mycn, Sox2, Klf2, Pou5f1, Sall4 and Esrrb (f, p<0.05, n=3).

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

According to a first aspect of the invention, there is provided a method of somatic cell nuclear reprogramming to alter the cell type comprising preparing a GV extract, permeabilising somatic cells, incubating the somatic cells with the GV extract to alter the cell type, and resealing somatic cell membranes, wherein said GV extract does not comprise oocyte cytoplasm. As such, factors present in the oocyte cytoplasm which may be undesirable for reprogramming are advantageously not present during downstream applications. Such factors are considered undesirable as they do not contribute to reprogramming, or are detrimental to reprogramming. Furthermore, as the non-desirable oocyte cytoplasm contaminants are not present, the desirable factors contained within the GV extract are incubated with the somatic cells at a higher concentration.

In one embodiment, the altering the cell type may comprise reprogramming the somatic cell to a progenitor cell or stem cells.

In one embodiment, the method of somatic cell nuclear reprogramming is an in vitro, in vivo, or ex vivo method.

In one embodiment, the progenitor cell is a unipotent stem cell, oligopotent stem cell, multipotent stem cell, pluripotent stem cell or totipotent stem cell.

As such, somatic cells incubated with GV extracts can be reverted to a progenitor, unipotent stem cell, oligopotent stem cell, multipotent stem cell, pluripotent stem cell or totipotent stem cell.

In one embodiment, the method may further comprise the differentiation of the progenitor cell and stem cell to alter the cell type.

In one embodiment the differentiation of the progenitor cell and stem cells may produce a somatic cell or progenitor thereof, wherein the somatic cell or progenitor thereof is of a different lineage.

In one embodiment, the differentiation may comprise the incubation of the progenitor cell and stem cell with differentiation medium. In one embodiment, the differentiation may comprise the addition of one or more differentiation factors. Various methods of differentiating progenitor cells or stem cells so as to produce somatic cells and/or their progenitors are available and known to a person skilled in the art. Such established methods are considered routine and are in no way a limitation to the present invention. For example, where the progenitor cell or stem cell is a pluripotent or totipotent cell, ectoderm, mesoderm and endoderm may be produced³⁷.

In the presence of differentiation factors these progenitors or stem cells can be differentiated into a completely different cell type from the original cells. Therefore, somatic cell nuclear reprogramming is achievable to alter the cell type when incubated in the presence of GV extract and other reprogramming factors together with one or more differentiation factors. Altering the cell type has many applications in both research and clinical settings.

The present invention in one embodiment may therefore comprise the step of creating a progenitor cell or stem cells following incubation of the somatic cell with the GV extract and differentiating said progenitor cell or stem cell to alter the cell type.

The present invention of altering the cell type of a cell may be performed in a single step. Reverting the cells to a progenitor or stem cell state or transdifferentiating cells to another differentiated state, may be performed simultaneously by incubating the cells with GV extract and other differentiation factors at the same time. The present invention may also be performed in two stages 1) reverting the cells to a progenitor or stem cell state and 2) after the cells have reached a progenitor or stem cell state, adding differentiation factors to differentiate the cells to a new cell type.

The present invention may therefore have applications for personalised medicine and regenerative medicine. Using the present invention, it is possible to generate embryonic stem cells which can be used to create different cell types to treat numerous diseases and dysfunctions in humans and animals. For example, the cells with an altered cell type generated using the present invention may be used to create healthy cells, tissues, or organs to assist in restoring normal function to a human or animal body.

In one embodiment, the reprogramming factors are factors which change chromatin accessibility. The reprogramming factors may be DNA modifying enzymes, histone variants, histone-modifying enzymes, chromatin remodelers, chromatin modifiers and transcription factors.

In some embodiments, the DNA modifying enzymes are AID or Mbd3. In some embodiments the histone-modifying enzymes are kdm4 or kdm6. In some embodiments, the chromatin remodelers are Brg1. In some embodiments, the transcription factors are Gli1, FoxA, Gata4, Ascl1, Brn2, Myt1l, PU.1, or xklf2.

In one embodiment, the isolated GV is derived from a meiotic oocyte. In one embodiment, the GV may be isolated from a Xenopus or mammal or a ray-finned fish. In one embodiment, the GV may be derived from a mouse, pig, cattle, human, salmon, sturgeon or Xenopus oocyte. In one embodiment, the GV may be derived from a human oocyte. In one embodiment, the GV may be derived from a salmon or sturgeon oocyte. In one embodiment, the GV may be derived from a Xenopus oocyte.

GV extract can be used to revert any cell type to a progenitor or stem cell state and the reprogramming factors used to alter the identity of the cell to a different cell type. This will allow, for example, skin cells to be taken from an individual and altered into neurons, which can be returned to the same individual without any rejection to the implanted cells.

In one embodiment, the altering the cell type may further comprise the addition of one or more exogenous reprogramming factors. The exogenous reprogramming factors may be produced recombinantly. In one embodiment the reprogramming factors may be incubated with the somatic cells before, simultaneously, or after the incubation with the GV extract.

In some embodiments, the exogenous reprogramming factors may be DNA modifying enzymes, histone variants, histone-modifying enzymes, chromatin remodelers, chromatin modifiers and transcription factors of the invention, or may be other factors. As such, the incubation of the somatic cells and the GV extract may contain further factors. In some embodiments, the incubation of the somatic cells and the GV extract is in the absence of exogenous reprogramming factors.

While it will be appreciated that incubation of the somatic cells and the GV extract, and optionally one or more exogenous reprogramming factors, may be performed for any time period required to achieve alteration of the cell type, preferred time periods in certain embodiments may be any one of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours. Alternatively, preferred periods of time in certain embodiments may be any one of about 1, 2, 3, 4, 5, 6, 7, 8, 9, or 10 days.

In one embodiment, the progenitor cell or stem cell may be incubated for any one of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days in differentiation medium or 1.5, 2, 3, 4, 5, 6, 7, 8, 9, 10, 11, 12 months in differentiation medium. As referred to above, various methods of differentiating progenitor cells or stem cells so as to produce somatic cells and/or their progenitors are available and known to a person skilled in the art. Such established methods are considered routine and are in no way a limitation to the present invention. As such, the invention extends to variations of the above incubation durations.

In one embodiment, the somatic cells may comprise a selectable or reportable marker, which may comprise totipotency genes, which may comprise one or more of Ne1fa, Top2a, Gata2, Eif3h, Dppa2/4, and Atr, pluripotency genes, which may comprise one or more of Jun, Sox2, Myc, Klf4, Mycn, Klf2, Pou5f1, Utf1, and Sall4, which may comprise trophoblast genes which may comprise one or more of Tfap2c, Hand1, Msx2, Csf1r, and Gcm1, and which may comprise gene markers for somatic cells, comprising Neun, Tubb3, Myod1, Myog, and Itga7. Markers advantageously allow for the cells to be monitored and selected for during the method.

The somatic cells may be permeabilised using Streptolysin O, digitonin, lysolecithin, or mixtures thereof. Permeabilization is an important step in the process to allow the factors from the GV extract to enter the cells and cause nuclear reprogramming. Beyond the agents listed above, any known permeabilization agent and transfection reagents may be used in the context of the present invention.

The cell membranes are resealed using CaCl₂. Calcium may be used to reseal the membranes after completion of the method. The use of calcium chloride is particularly advantageous for resealing cells.

In one embodiment, the step of preparing a GV extract may comprise mechanical dissociation. In one embodiment, the step of mechanical dissociation may comprise centrifugation. The step of mechanical dissociation may occur in mineral oil.

In one embodiment, the method may further comprise a step of modifying the GV extract to express a gene encoding a protein selected from the list comprising Gli1, FoxA, Gata4, Ascl1, Brn2, Myt1l, PU.1, xklf2, xsox2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof. In one embodiment, the GV extract may overexpress a gene encoding a protein selected from the list comprising Gli1, FoxA, Gata4, Ascl1, Brn2, Myt1l, PU.1, xklf2, xsox2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof. As used herein, ‘x’ designates ‘Xenopus’, ‘h’ designates ‘human’ and ‘m’ designates ‘mouse.’

In one embodiment, the step of modifying the GV extract may comprise injecting the cytoplasm with mRNA or incubating the oocyte with a rare-cutting endonuclease, for example a TALEN, ZFN or CRISPR/Cas9 and guide RNA. In one embodiment, the mRNA may encode a protein selected from the list comprising Gli1, FoxA, Gata4, Ascl1, Brn2, Myt1l, PU.1, xklf2, xsox2, xklf2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof. In one embodiment, the guide RNA may comprise a gene encoding Gli1, FoxA, Gata4, Ascl1, Brn2, Myt1l, PU.1, xklf2, xsox2, xklf2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof.

Therefore, the GV extract may be modified to express or overexpress certain factors which may be useful in reprogramming somatic cells. These factors may include the factors identified above. The method in which such modified occurs are not limited to a specific technique; suitable techniques include for example, injecting a GV with mRNA encoding such factors or may be by genetic modification by a suitable technique, such as CRISPR Cas9 gene editing. This has the advantage of increasing the concentration of desirable reprogramming factors in the GV extract. Conversely, modification of the GV may involve the reduction or silencing of inhibitors of reprogramming. For example, siRNA may be injected into the GV extract so as to reduce or silence inhibitors of reprogramming.

The step of modifying the GV extract may occur before the incubation of the GV extract with the somatic cells. For example, the step of modifying the GV extract may occur approximately 24 hours before the incubation of the GV extract with the somatic cells. Optionally, the GV extracts may be tested for the expression of the exogenous factors by routine methods (for example qPCR) prior to incubation with the somatic cells.

In one embodiment, reprogramming factors may be identified using any suitable method. Examples include, but are not limited to, experimental screening, computational methods and artificial intelligence. For example, methods such as DeepAccess and diffTF as identified in Hammelman et al³⁸ may be used.

In a further aspect of the invention, there is provided a GV extract comprising reprogramming factors. In one embodiment, the GV may be derived from mouse, pig, cattle, human, salmon, sturgeon or Xenopus oocytes. In one embodiment, the GV may be derived from human, mouse, pig, cattle oocytes. In one embodiment, the GV may be derived from salmon, or sturgeon oocytes. In one embodiment, the GV may be derived from Xenopus oocytes. In one embodiment, the GV may be derived from a Xenopus or mammal or a ray-finned fish.

In one embodiment, the reprogramming factors are factors which change chromatin accessibility. The reprogramming factors may be DNA modifying enzymes, histone variants, histone-modifying enzymes, chromatin remodelers, chromatin modifiers and transcription factors.

In some embodiments, the DNA modifying enzymes are AID or Mbd3. In some embodiments, the histone-modifying enzymes are kdm4 or kdm6. In some embodiments, the chromatin remodelers are Brg1. In some embodiments, the transcription factors are Gli1, FoxA, Gata4, Ascl1, Brn2, Myt1l, PU.1, xklf2, xsox2, xklf2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof.

In a further aspect of the invention, there is provided a cell derived from the method of the first aspect of the invention.

In a further aspect of the invention, there is provided a cell with an altered cell type derived from the method of the first aspect of the invention.

In yet a further aspect of the invention, there is provided a kit comprising a permeabilization agent, a GV extract and/or one or more reprogramming factors, and optionally instructions for use. In one embodiment, the kit may further comprise a membrane resealing agent. In one embodiment the membrane resealing agent may be CaCl₂.

The term “GV extract” as used herein refers to germinal vesicle (GV) extracted from an oocyte. The GV extract may comprise whole GV or a lysate of GV. In one embodiment, the GV extract may comprise further components, such as, common factors found in growth media. In other embodiments, the GV extract does not comprise further components, such as, common factors found in growth media or protease inhibitors. In a further embodiment, the media may be CA2+ free media. In one embodiment, the GV may be derived from Xenopus oocytes. GV may be dissected manually in the mineral oil. In one embodiment, GV extracts may be derived by snap freeze thawing dissected GV or mechanical disruption of the GV, for example pipetting the GV up and down to rupture the dissected GV. In one embodiment, the GV extract may be filtered prior to use. As will be appreciated, whole GV or lysate of GV contains multiple factors which are useful for reprogramming.

The term “totipotent” as used herein is a cell capable of giving rise to any cell type or a complete organism.

As used herein, the term “pluripotent cell” or “pluripotent stem cell” (PSCs) refers to a cell that has complete differentiation versatility, e.g., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), a pluripotent cell, even a pluripotent embryonic stem cell, cannot usually form a new blastocyst.

As used herein, the term “multipotent cell” refers to a cell that can differentiate into a limited number of specialised cell types. As used herein, the term “oligopotent cell” refers to a cell that can differentiate into a more limited number of specialised cell types. As used herein, the term “unipotent cell” refers to a cell that can differentiate into one lineage.

As used herein, the term “progenitor cell” refers to a lineage-restricted cell which has limited proliferation capacity.

The term “cell type” as used herein is a classification used to identify cells that share the same morphological or phenotypical identity.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The term “comprising” or “comprises” where used herein means including the component(s) specified but not to the exclusion of the presence of other components. The term “consisting essentially of” or “consists essentially of” means including the components specified but excluding other components except for materials present as impurities, unavoidable materials present as a result of processes used to provide the components and the like.

The term “consisting of” or “consists of” means including the components specified but excluding other components.

Whenever appropriate, depending upon the context, the use of the term “comprises” or “comprising” may also be taken to include the meaning “consists essentially of” or “consisting essentially of”, and also may also be taken to include the meaning “consists of” or “consisting of”.

The optional features set out herein may be used either individually or in combination with each other where appropriate and particularly in the combinations as set out in the accompanying claims. The optional features for each aspect or exemplary embodiment of the invention, as set out herein are also applicable to all other aspects or exemplary embodiments of the invention, where appropriate. In other words, the skilled person reading this specification should consider the optional features for each aspect or exemplary embodiment of the invention as interchangeable and combinable between different aspects and exemplary embodiments.

### Clauses

1. A method of nuclear reprogramming somatic cells to alter the cell type comprising: preparing a GV extract;
permeabilising somatic cells;
incubating the somatic cells with the GV extract to alter the cell type; and
resealing somatic cell membranes,
wherein said GV extract does not comprise oocyte cytoplasm.
   2. The method according to clause 1, wherein the altering the cell type comprises reprogramming the somatic cell to a progenitor cell or stem cell.
   3. The method according to clause 2, wherein the progenitor cell or the stem cell is a unipotent stem cell, oligopotent stem cell, multipotent stem cell, pluripotent stem cell or totipotent stem cell.
   4. The method according to any of clauses 2 or 3, further comprising the differentiation of the progenitor cell or stem cell to alter the cell type.
   5. The method according to clause 4, wherein the differentiation of the progenitor cell or stem cell produces a somatic cell or progenitor thereof, wherein the somatic cell or progenitor thereof is of a different lineage.
   6. The method according to clauses 4 or 5, wherein the differentiation comprises the incubation of the progenitor cell or stem cell with differentiation medium.
   7. The method according to clause 6, wherein the differentiation medium further comprises the addition of one or more differentiation factors.
   8. The method according to any of the preceding clauses, wherein the GV extract comprises one or more reprogramming factors.
   9. The method according to clause 8, wherein the reprogramming factors are factors which change chromatin accessibility.
   10. The method according to clause 9, wherein reprogramming factors are DNA modifying enzymes, histone variants, histone-modifying enzymes, chromatin remodelers, chromatin modifiers and transcription factors.
   11. The method according to clause 10, wherein the DNA modifying enzymes are AID or Mbd3.
   12. The method according to clause 10, wherein the histone-modifying enzymes are kdm4 or kdm6.
   13. The method according to clause 10, wherein the chromatin remodelers are Brg1.
   14. The method according to clause 10, wherein the transcription factors are Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof.
   15. The method according to any of the preceding clauses, wherein the isolated GV is derived from a meiotic oocyte.
   16. The method according to any of the preceding clauses, wherein the GV is isolated from a Xenopus, mammal or ray-finned fish.
   17. The method accord to clause 16, wherein the mammal is a human, mouse, pig or cattle and the ray-finned fish is a salmon or a sturgeon.
   18. The method according to any of the preceding clauses, wherein the somatic cells are mammalian cells.
   19. The method according to clause 18, wherein the mammalian cells are human cells, mouse cells, pig cells, cattle cells, dog cells, cat cells or horse cells.
   20. The method according to any of the preceding clauses, wherein the altering the cell type further comprises the addition of one or more exogenous reprogramming factors.
   21. The method according to any of the preceding clauses, wherein the somatic cells and the GV extract, and optionally one or more exogenous reprogramming factors, are incubated for any one of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours.
   22. The method according to any one of clauses 1 to 20, wherein the somatic cells and the GV extract are incubated for any one of about 1, 2, 3, 4, 5, 6, 7, 8, 9, or 10 days.
   23. The method according to any one of clauses 6 to 22, wherein the progenitor cell is incubated for any one of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 days in differentiation medium or 1.5, 2, 3, 4, 5, 6, 7, 8, 9, 10, 11, 12 months in differentiation medium.
   24. The method according to any preceding clauses, wherein the somatic cells comprise a selectable or reportable marker, comprising totipotency genes, comprising one or more of Ne1fa, Top2a, Gata2, Eif3h, Dppa2/4, and Atr, pluripotency genes, comprising one or more of Jun, Sox2, Myc, Klf4, Mycn, Klf2, Pou5f1, Utf1, and Sall4, comprising trophoblast genes comprising one or more of Tfap2c, Hand1, Msx2, Csf1r, and Gcm1, and comprising gene markers for somatic cells, comprising Neun, Tubb3, Myod1, Myog, and Itga7.
   25. The method according to any one of the preceding clauses, wherein the somatic cells are permeabilised using Streptolysin O, digitonin, lysolecithin, or mixtures thereof.
   26. The method according to any one of the preceding clauses, wherein the cell membranes are resealed using CaCl₂.
   27. The method according to any of the preceding clauses, wherein the step of preparing a GV extract comprises mechanical dissociation.
   28. The method according to any of the preceding clauses, wherein the method further comprises a step of modifying the GV extract to express a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof.
   29. The method according to any of the preceding clauses, wherein the GV extract overexpresses a gene encoding a protein selected from the list comprising Gli1, FoxA, Gata4, Ascl1, Brn2, Myt1l, PU.1, xklf2, xsox2, xklf2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof.
   30. The method according to clause 28 or clause 29, wherein the step of modifying the GV extract comprises injecting the cytoplasm with mRNA or incubating the oocyte with a rare-cutting endonuclease, for example a TALEN, ZFN or CRISPR/Cas9 and guide RNA, optionally, wherein the mRNA encodes a protein selected from the list comprising Gli1, FoxA, Gata4, Ascl1, Brn2, Myt1l, PU.1, xklf2, xsox2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof or the guide RNA comprises a gene encoding Gli1, FoxA, Gata4, Ascl1, Brn2, Myt1l, PU.1, xklf2, xsox2, xklf2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof.
   31. A GV extract comprising reprogramming factors, optionally, wherein the reprogramming factors are factors which change chromatin accessibility.
   32. The GV extract according to clause 31, wherein reprogramming factors are DNA modifying enzymes, histone variants, histone-modifying enzymes, chromatin remodelers, chromatin modifiers and transcription factors.
   33. The GV extract according to clause 32, wherein the DNA modifying enzymes are AID or Mbd3.
   34. The GV extract according to clause 32, wherein the histone-modifying enzymes are kdm4 or kdm6.
   35. The GV extract according to clause 32, wherein the chromatin remodelers are Brg1.
   36. The GV extract according to clause 32, wherein the transcription factors are Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof.
   37. The GV extract according to any one of clauses 31 to 36, wherein the GV is isolated from a Xenopus or a mammal or ray-finned fish.
   38. The GV extract according to clause 37, wherein the mammal is a human, mouse, pig, or cattle and the ray-finned fish is a salmon or a sturgeon.
   39. The GV extract according to any of clauses 31 to 38, wherein the GV extract is modified, optionally wherein the GV is modified to express a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, or homologues thereof.
   40. The GV extract according to clause 39, wherein the GV is modified to overexpress a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, or homologues thereof.
   41. A cell derived from the method of clauses 1 to 30.
   42. A cell with an altered cell type derived from the method of clauses 1 to 30.
   43. A kit comprising a permeabilization agent, a GV extract and/or one or more reprogramming factors, and optionally instructions for use.
   44. The kit according to clause 43, further comprising differentiation medium and/or one or more differentiation factors.
   45. The kit according to clauses 43 or 44, wherein the GV extract is modified, optionally wherein the GV is modified to express or overexpress a gene encoding a protein selected from the list comprising Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, or homologues thereof.

### Examples

The invention is further described in the following non-limiting examples.

### Example 1—Early Chromatin Changes and Nuclear Envelope Breakdown of MEF Nuclei Transplanted to Xenopus Oocytes

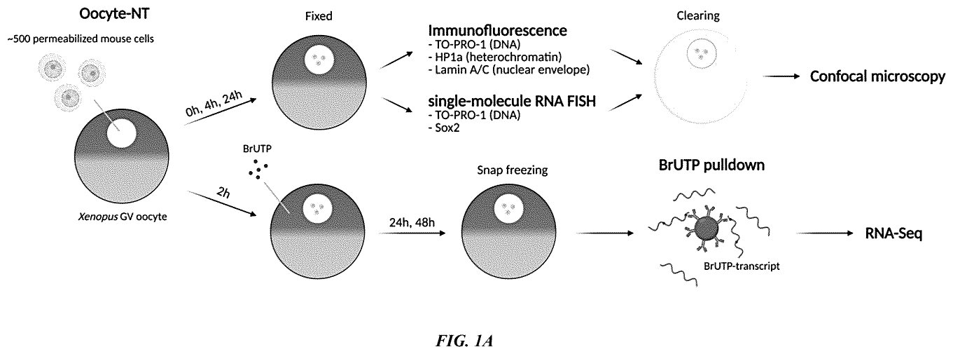

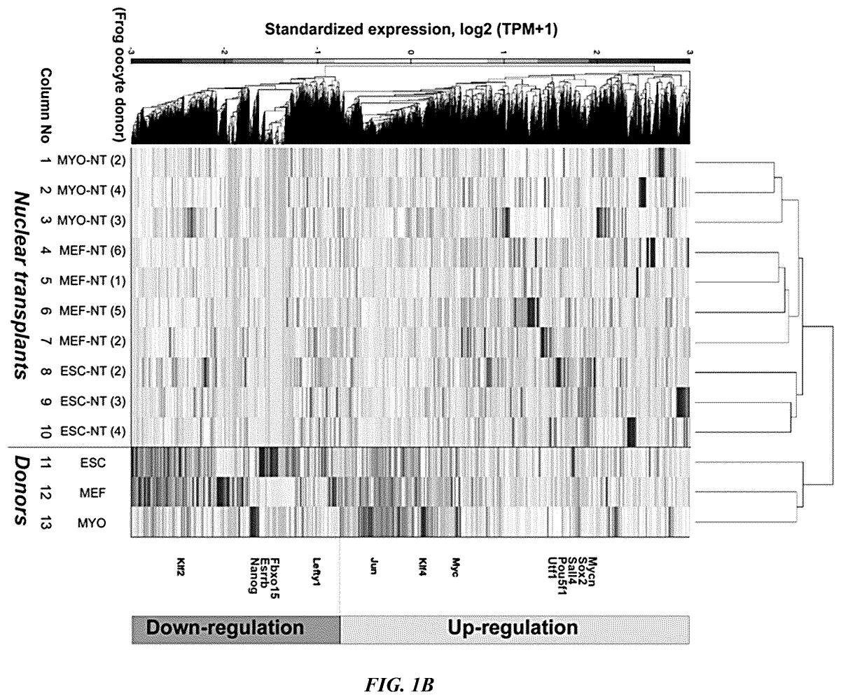

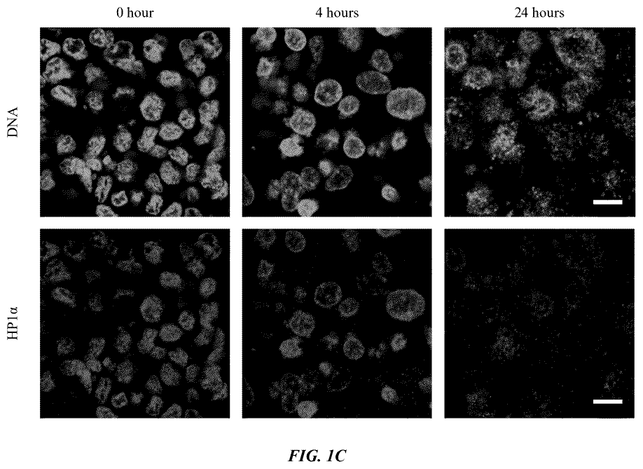

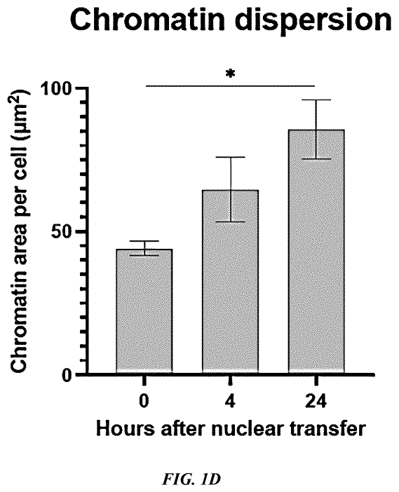

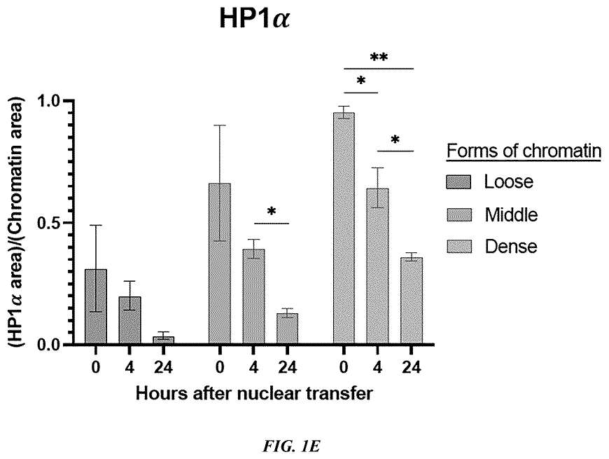

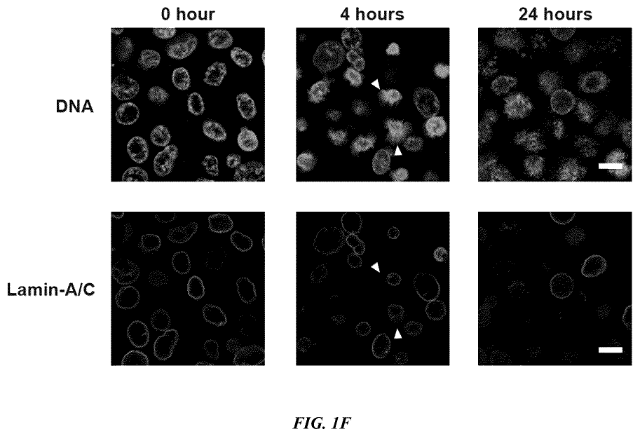

Our procedure here is as follows (FIG. 1A). About 500 nuclei in 10 nanolitres from ESC (mouse embryonic stem cells), MEF or MYO (mouse myoblasts) are injected into the GVs of oocytes, which are incubated for up to 48 hours for the following analyses. We have used several different females as donors for our oocytes. We have confirmed that the results we get are not affected by differences in the source of oocytes of one female compared to those of another. Whenever we see a difference in gene expression between one kind of donor nuclear preparation and another, we see the same difference with oocytes of other females. In later experiments for RNA-seq, six different females were used to supply recipient oocytes (FIG. 1B). There are substantial differences in gene expression when comparing donor nuclear preparations from ESCs, MEFs, and MYOs, but results with a given nuclear preparation are very similar with different sources of oocytes (FIG. 1B, compare columns 1-3 with 4-7 and 8-10). Our results are therefore not affected by the source of oocytes.

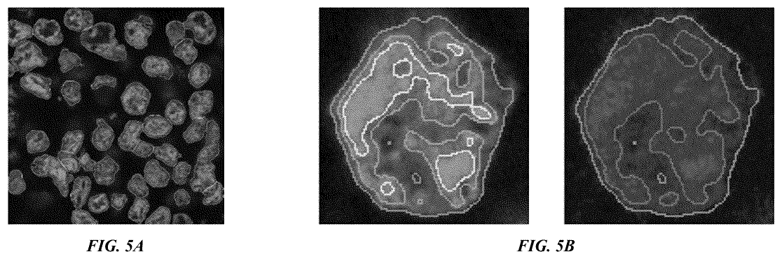

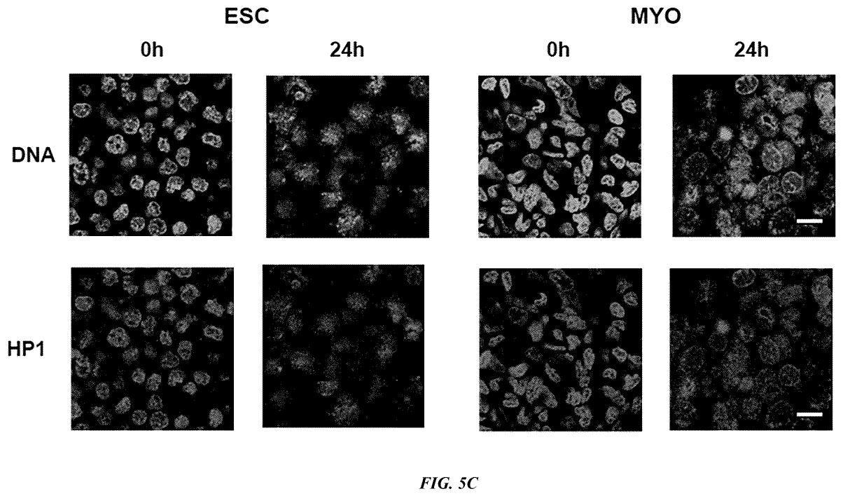

In earlier work on nuclei injected into oocytes, we saw significant nuclear enlargement and chromatin dispersal at Day 3 or 4 after nuclear transfer¹⁵. We now ask on what time scale these effects initially occur; do these effects precede or follow changes in gene expression? To quantify chromatin changes, the chromatin of transplanted MEF is marked by the DNA dye, TO-PRO-1, and heterochromatin protein 1 alpha, HP1α (FIG. 1C). Changes to chromatin structure in injected oocytes are seen in FIGS. 1C-1E. At 24 hours after MEF nuclear transfer, the chromatin of injected nuclei is about 3-fold dispersed in volume, judged by the 2-fold increase of chromatin area for each cell (FIGS. 1C-1D and FIG. 5A). The time-dependent reduction of HP1α in chromatin indicates the decondensation of heterochromatin (FIG. 1C, FIG. 1D, and FIG. 5B). This change shows that the dispersion of chromatin and decondensation of heterochromatin take place within 24 hours of nuclear transfer (FIGS. 1C-1E). These conformational changes of chromatin are also seen in ESC and MYO (FIG. 5C). We conclude that the dispersion and decondensation of chromatin begins with the early entry of GV factors in oocytes and is likely to reflect the gene expression changes induced by nuclear transfer to oocytes.

The drastic change of chromatin further raises the question of how the nuclear envelope of transplanted MEFs responds to the three-fold dispersion of chromatin after nuclear transfer. To answer this, we used anti-Lamin-A/C antibody to mark the nuclear envelope of transplanted MEFs and observed the change of the nuclear envelope accompanying the dispersion of chromatin (FIG. 1F). The nuclear envelope was intact at 0 hour (FIG. 1F); at 4 hours, the nuclear envelopes of transplanted MEFs started to break down (lower middle, white arrowhead, FIG. 1F). From the place where that took place, the chromatin contained in the donor nuclei burst out (upper middle, white arrowhead, FIG. 1F). At 24 hours after nuclear transfer, most of the nuclear envelopes of transplanted MEFs had disappeared and chromatin was dispersed (FIG. 1F).

Here, we show that HP1α is removed during oocyte reprogramming. It is known that HP1α is crucial for the higher-order structure of constitutive heterochromatin and binds to the H3K9me3 for gene repression and silencing^{16, 17}. Nuclear transfer of somatic nuclei to eggs and oocytes shows the resistance of genes is marked by H3K9me3, which is alleviated by ectopic H3K9me3 demethylase Kdm4d^{18, 19}. Furthermore, we show the breakdown of nuclear envelope happens during oocyte reprogramming, accompanying the disappearance of Lamin-A/C, which constitutes the nuclear lamina with other intermediate filaments. The Lamin-A/C-composed nuclear lamina is tethered to HP1-H3K9me3-marked peripheral heterochromatin and functionally linked to gene repression/silencing^{20, 21}. In conclusion, the removal of HP1α and the disappearance of Lamin-A/during oocyte reprogramming indicate the forced disassembly of H3K9me3-dependent heterochromatin and may lead to the transcriptional reprogramming after nuclear transfer.

### Example 2 Completion of Transcriptional Reprogramming to a Totipotency-Like State by Xenopus Oocytes within 24 Hours

The nuclear membrane breakdown and chromatin changes are similar in nuclear transfers to eggs and oocytes. Are the transcriptional changes also similar in both types of nuclear transfers? We have measured the transcriptional changes of oocyte-induced genes in donor cell nuclei via BrUTP incorporation after nuclear transfer. We determine the newly synthesized BrUTP-transcripts in the 24-hour and 48-hour period after nuclear transfer and compare these BrUTP-transcripts to the published RNA-seq data for gene expression patterns of normal cultured cells from ESC, MEF and MYO. These published results came from the Expression Atlas, EMBL-EBI, E-GEOD-27843 for ESC and MEF; for MYO, they were from ENCODE, ENCSR000AHY. The comparison between donor cells and nuclear transplants is summarised in Table 1.

TABLE 1

| | 0-24 hour | | | 0-48 hour | | |
|---|---|---|---|---|---|---|
| Donor Cell Type | Nuclear transfer change [symbol] | Gene number | %^{†} | Nuclear transfer change | Gene number | % |
| MEF | Enhancement | 2333 | 10% | Enhancement | 2357 | 10% |
| | Activation | 3864 | 16% | Activation | 2381 | 18% |
| | Reduction | 2353 | 10% | Reduction | 2490 | 11% |
| | Silencing | 1396 | 6% | Silencing | 919 | 4% |
| | Overall | 9946 | 42% | Overall | 10147 | 43% |
| ESC | | | | Enhancement | 2314 | 10% |
| | | | | Activation | 2926 | 12% |
| | | | | Reduction | 2484 | 11% |
| | | | | Silencing | 1674 | 7% |
| | | | | Overall | 9398 | 40% |
| MYO | | | | Enhancement | 2564 | 11% |
| | | | | Activation | 4237 | 18% |
| | | | | Reduction | 1228 | 5% |
| | | | | Silencing | 902 | 4% |
| | | | | Overall | 8931 | 38% |

[symbol]

Enhancement means increase of transcripts from [symbol]
Activation means increase of transcripts from [symbol]
Reduction means decrease of transcripts to [symbol]
Silencing means decrease of transcripts to [symbol]
^{†}% of induced mouse genes by [symbol] [symbol]
indicates data missing or illegible when filed

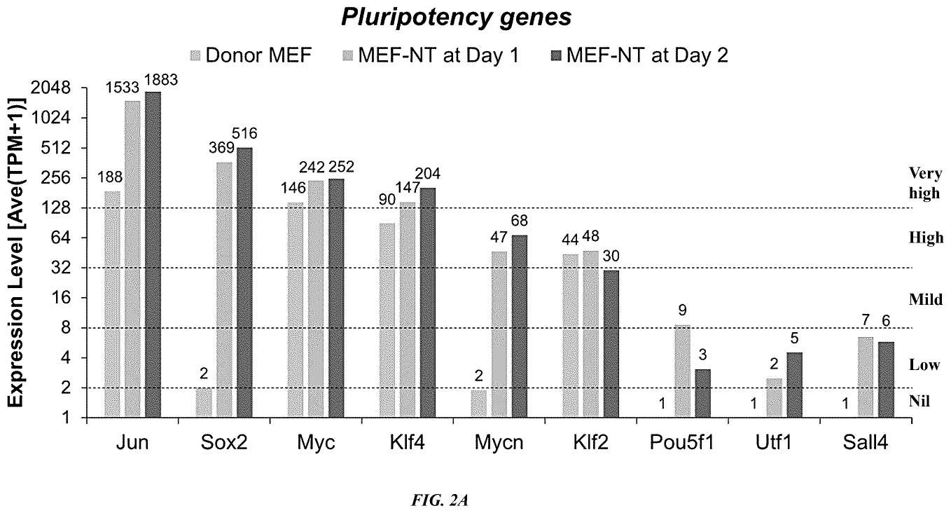

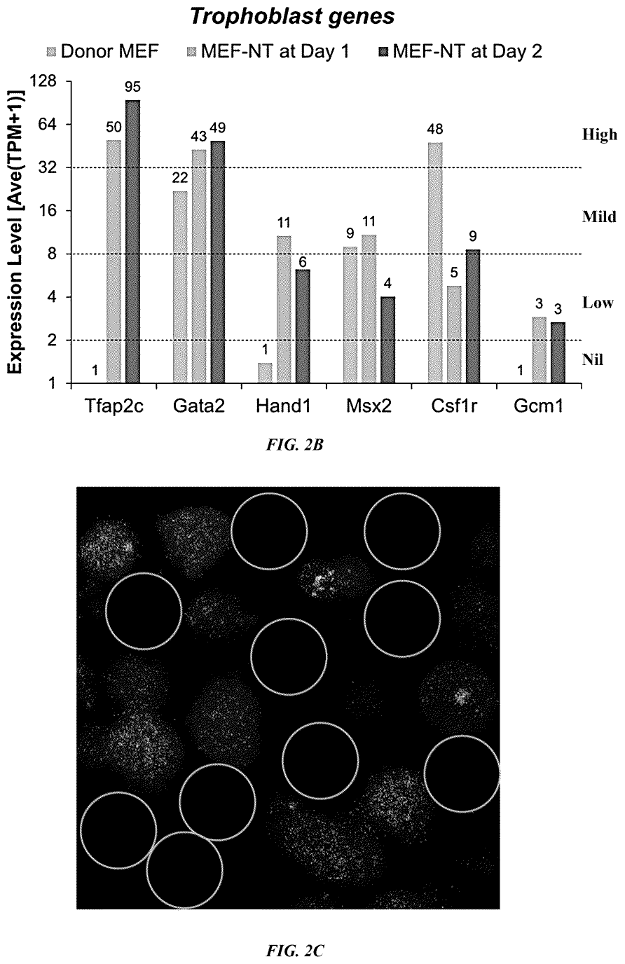

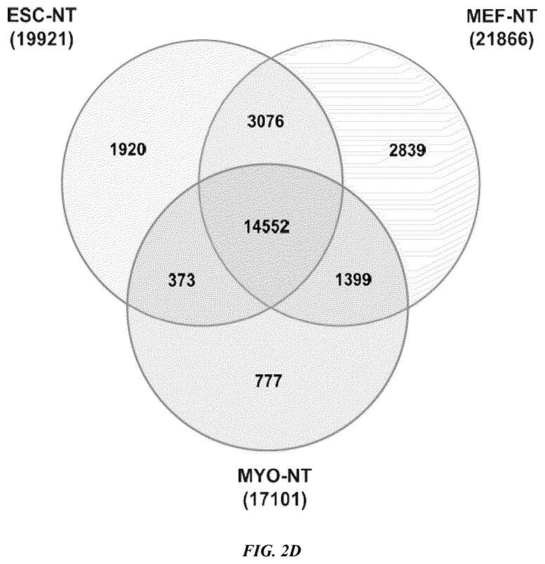

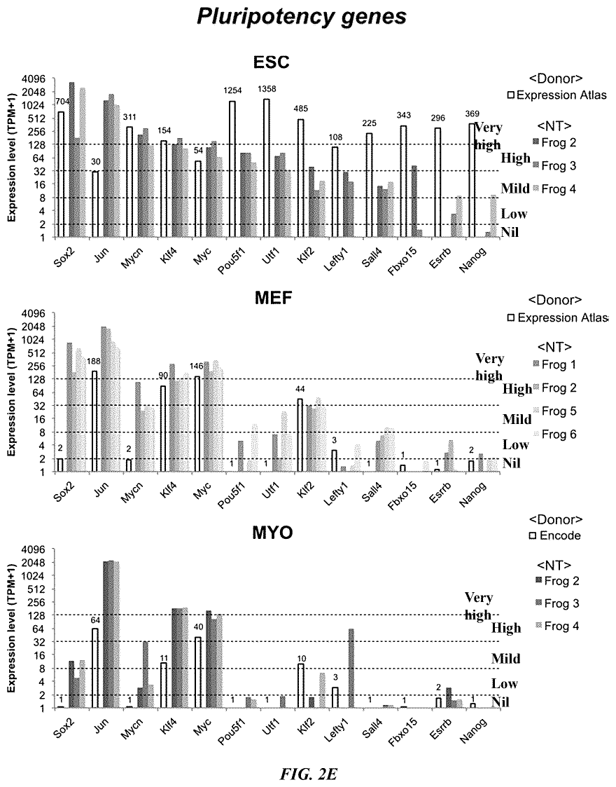

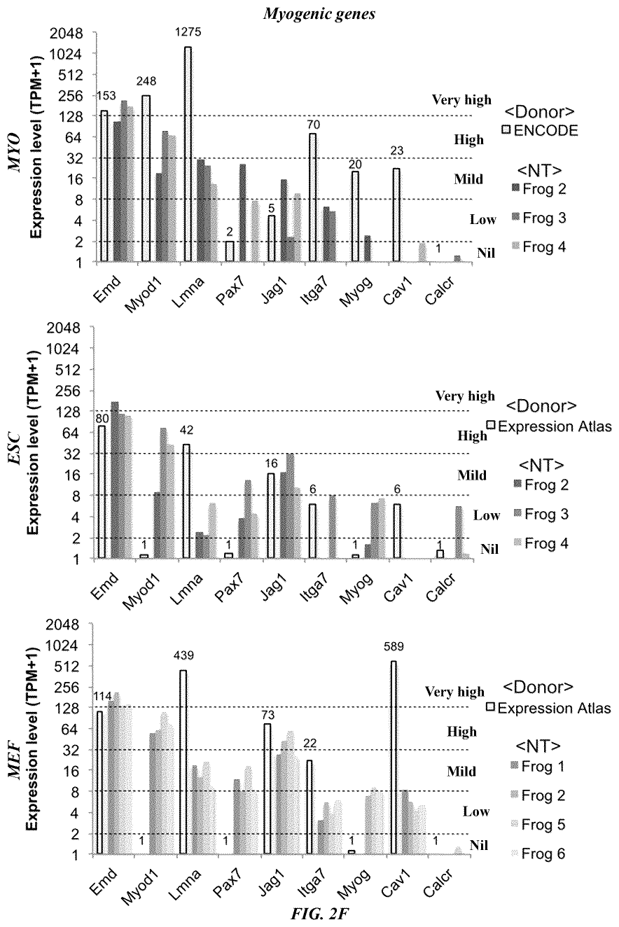

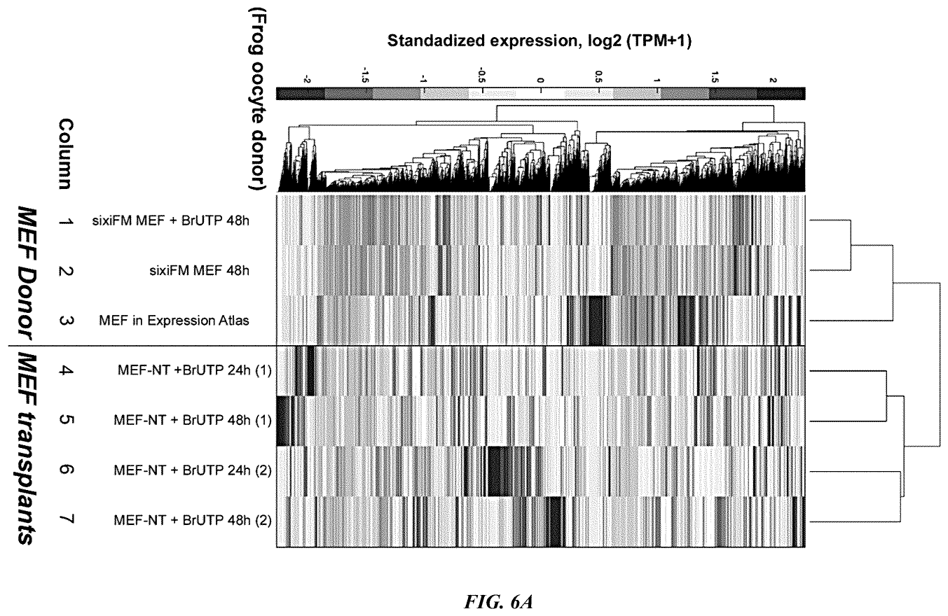

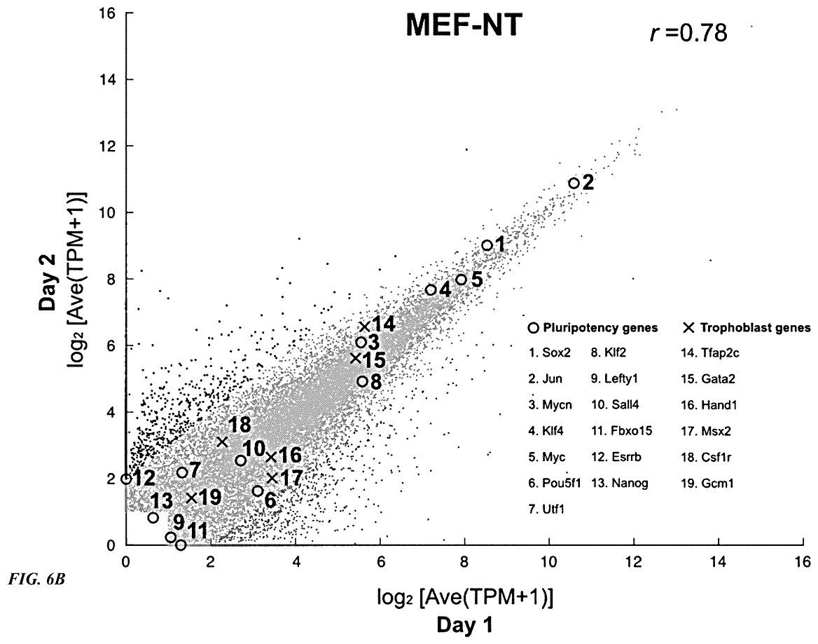

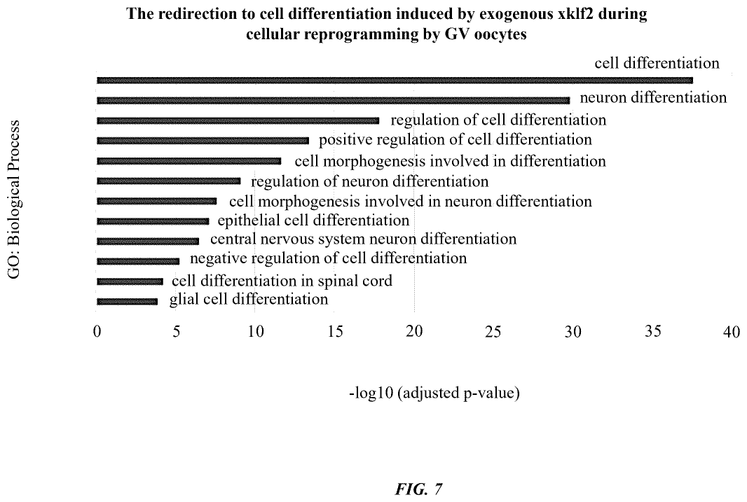

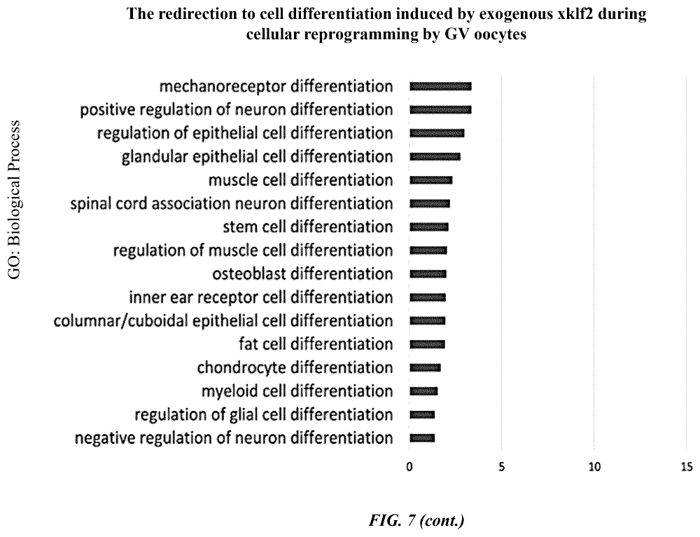

From our experiments, there are 29,195 genes whose transcript sequences are distinguishable for Xenopus laevis versus Mus musculus genomes (see Materials and Methods). In about 30% of the MEF genes analysed (6197/23525), there was an increase in transcripts after mouse nuclear transfers to Xenopus oocytes over 24 hours and in others a reduced expression was seen (Table 1). Therefore, about 40% of the MEF genes were induced to change transcription, up or down, by the combined effects of the oocyte components, and no difference between samples incubated for 24 and 48 hours was seen after nuclear transfer (Table 1). These similar changes were seen in ESC and, in nearly as many, in MYO (Table 1). Global gene expression also shows that oocyte components change the gene expression pattern in donor MEF to an oocyte-induced gene expression state that is similar at 24 and 48 hours after nuclear transfer (FIG. 6A and FIG. 6B). Most of these changes in gene expression took place in the first 24 hours after nuclear transfer as seen for MEF cells, including pluripotency genes and trophoblast genes (FIG. 2A and FIG. 2B). We, therefore, conclude that oocytes induce a totipotency-like state in transplanted MEF within 24 hours after nuclear transfer, and to this extent, gene expression changes induced in eggs and oocytes are similar.

The rapidity and extent of these changes in gene expression is very pronounced compared to what is believed to take place in iPSC experiments^{8, 22}. The difference is apparently even greater if we allow for the fact that oocyte nuclear transfers are cultured at 18° C. for amphibia compared to 37° C. for mammals. We have considered the possibility that only a very few of injected nuclei in the oocytes may progress to the stage when pluripotency genes are expressed. This is what happens in iPSC experiments by comparison. In iPSC experiments, usually only a small percentage of the DNA-transfected adult cells are switched to pluripotency, to behave like embryos. Unequal cell division and selection during the time when DNA-transfected cells are grown means that the majority of the DNA-transfected cells do not progress to a pluripotent, ESC-like state. Does the low percentage response also happen with nuclei-injected oocytes?

We have tested this by doing in situ hybridization with probes for Sox2 and we ask what proportion of the injected nuclei contain Sox2 transcripts. Since there is no replication or division of nuclei injected to oocytes, we hope that a high % of the injected nuclei would contain pluripotency gene transcripts, indicating that a high proportion of them will have been converted to pluripotency. The results are shown in FIG. 2C, from which it can be seen that nearly all, or perhaps all, of the injected and analysed nuclei contain some transcripts of Sox2 (marked in white, FIG. 2C). This means that after transfer to oocytes, the great majority of nuclei are converted to pluripotency.

We have seen that, from a global point of view, there is a strong similarity in the three donor nuclear types in how they respond to transplantation to oocytes (FIG. 1B, Table 1). We now ask if this similarity extends to the selection of individual genes that are expressed in these cell-types. There is a considerable, but not complete, overlap in gene expression by the three donor cell types (FIG. 2D, 14,552 in the total 24,936 oocyte-inducible genes, see also Table 2).

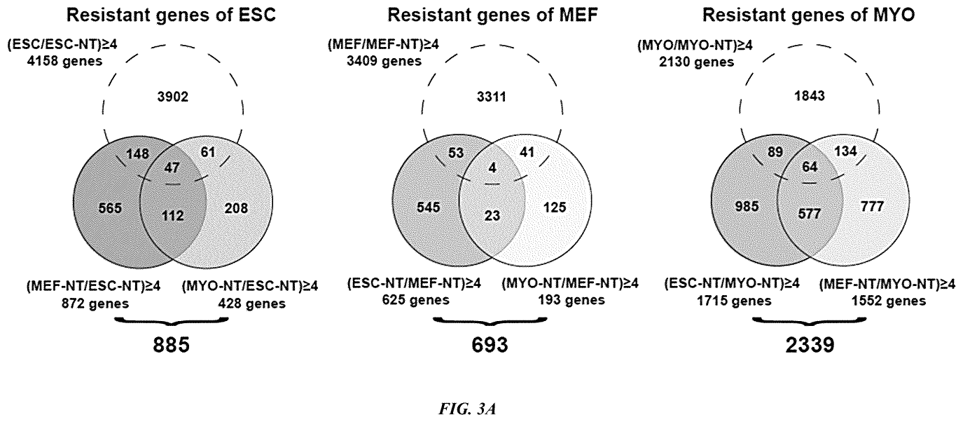

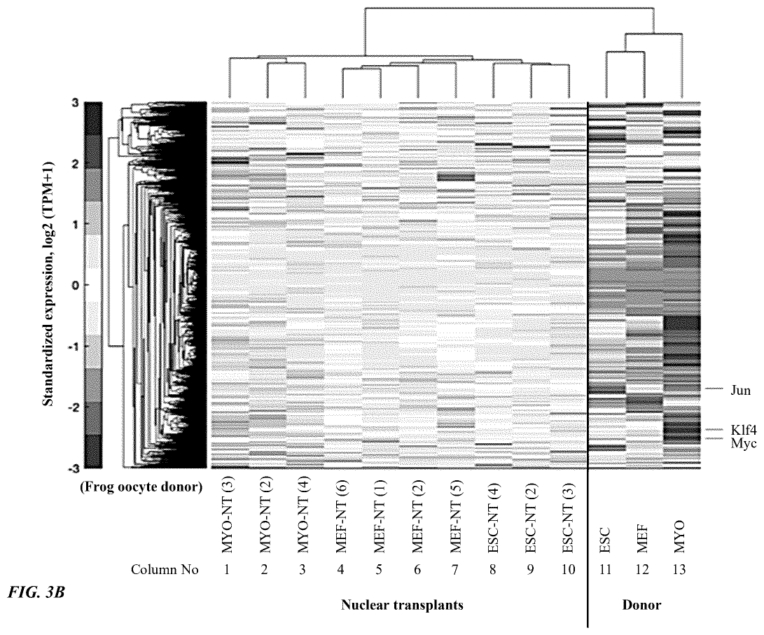

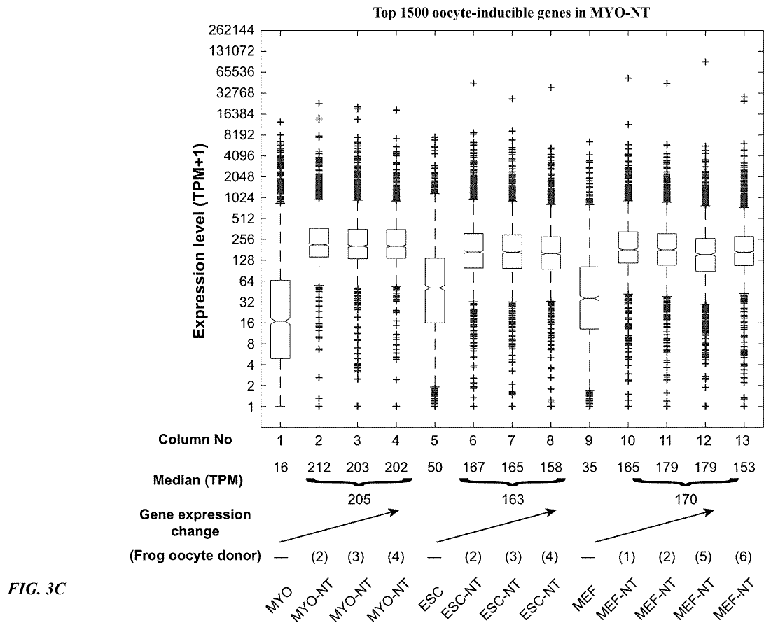

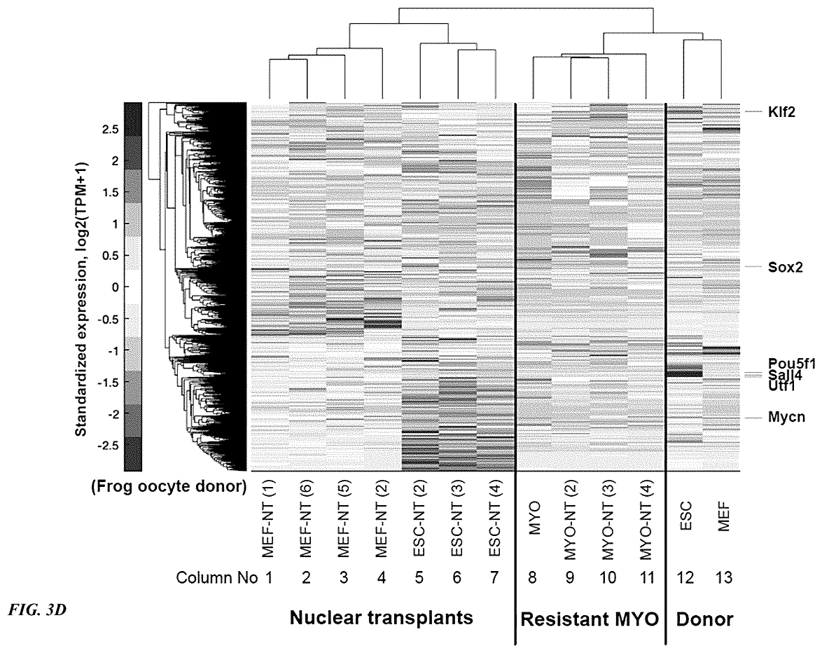

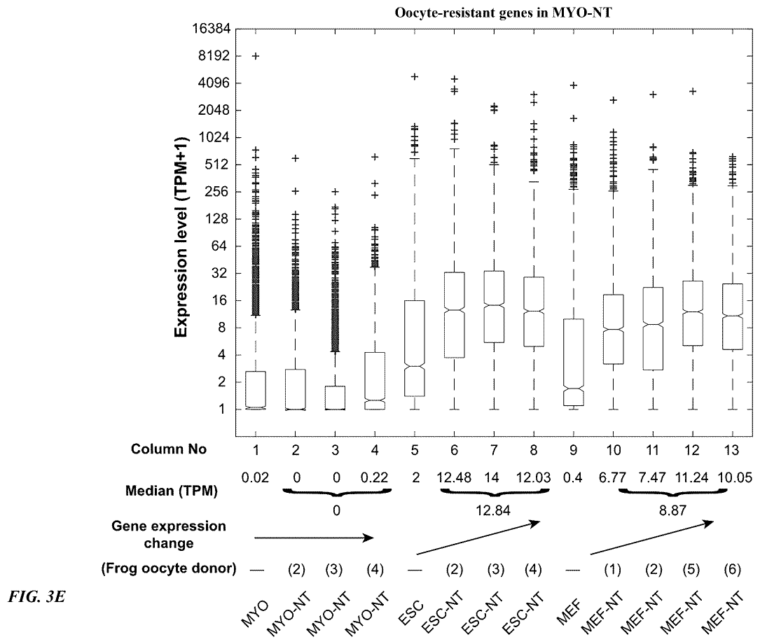

TABLE 2

| | ESC | MEF | MYO | |
|---|---|---|---|---|
| Numbers of inducible genes | 19,921 | 21,866 | 17,101 | FIG. 2d |
| Numbers of resistant genes | 885 | 693 | 2339 | FIG. 3a |

Examples of individual gene expression values are shown for pluripotency genes in FIG. 2E. We see that the further donor cells are from a differentiated state, the higher is the proportion of their genes that is activated, that is for ESC, MEF, and MYO in that order. Many of the most strongly activated genes are present in all three donor cell types, e.g., Jun, Klf4, and Myc, as the cells become more differentiated. If we limit our attention to individual myogenic gene transcript values, the results are similar for all three donor cell-types and most myogenic genes decrease in expression after nuclear transfer (FIG. 2F). Therefore, Xenopus oocytes induced the global changes in transplanted nuclei to a totipotency-like state and repress the cell type specific genes that represent a differentiated state within 24 hours after nuclear transfer, as happens in nuclear transfer to egg transplants.

### 3. Retention of Gene Resistance to Reprogramming in Differentiated Mouse Myoblasts after Nuclear Transfer

As cells approach a differentiated state, their nuclei become increasingly resistant to reprogramming. This was already known from the earliest nuclear transfer in amphibian egg experiments²³ (King and Briggs, 1955). We now ask if the same phenomenon can also be seen in somatic cell nuclear reprogramming by Xenopus oocytes. In the three donor cell types used here, this gene resistance is also seen in the present nuclear transfer to oocyte experiments which take place in the absence of cell division (Table 2). In our present results (FIG. 3A), nuclei of the more differentiated MYO nuclei have a greater number of resistant genes (2339 genes), compared to ESC (885 resistant genes), and MEF (693 resistant genes).

The transcriptional changes for oocyte-inducible genes in MYO are shown by heatmap and boxplot (FIG. 3B and FIG. 3C). The top 1500 most expressed oocyte-inducible genes in MYO nuclear transplants are up-regulated strongly by oocyte factors for all three cell types (FIG. 3B, columns 11-13 versus columns 1-10, blue to red). In the donor cells, these genes have mild to high expression levels (FIG. 3C, medians, 16 for MYO; 50 for ESC; 35 for MEF). After nuclear transfer, these genes are induced by oocytes to a very high expression level of similar median values for the three cell types with different fold changes because the initial expression levels are different between donor cell types (FIG. 3C). In contrast, the transcriptional changes of oocyte-resistant genes of MYO (FIG. 3D, columns 8-11) differ greatly from the same gene set for ESC and MEF which are mostly reprogrammed by oocyte factors successfully (FIG. 3D, columns 1-7 and 12-13). Therefore, our results establish a great resistance, the converse of nuclear reprogramming, of differentiating MYO to the reprogramming factors of oocytes with no such effects on ESC or MEF.

In the three donor cell types, most oocyte-inducible genes respond to oocyte factors similarly (FIG. 3C). However, there is a difference in oocyte-induced expression for those genes resistant to oocyte reprogramming (FIG. 3E). There is, therefore, a big difference in how MYO nuclei behave, after nuclear transfer, compared to ESC or MEF nuclei. MYO nuclei retain more resistance to oocyte reprogramming after nuclear transfer (2339 genes); ESC and MEF nuclei are less resistant to oocyte reprogramming for fewer resistant genes (885 and 693 genes, FIG. 3A). Overall, we conclude that the process of cell differentiation is accompanied by a progressive resistance to oocyte induced reprogramming, as seen in both egg nuclear transfer and oocyte nuclear transfer experiments.

### Example 4. Potential Application to Humans

We have pursued the idea that nuclear reprogramming to a totipotent-like state by GV extracts of frog meiotic oocytes could be beneficial for human therapy. This could certainly be the case if adult human cells of different kinds come to lose their normal function, as can happen with age and disease. Following an earlier report²⁴, two recent papers have described derivation of human pluripotent cells, one by Yamanaka factors and one by chemicals^{25, 26} Human fibroblasts can be grown from most human adult tissues. Nucleated human blood cells, such as lymphocytes and human blood stem cells, are readily obtained and turned into growing cultures, as is done for many other cell types. These cultured cell types can be reprogrammed by our GV-extract procedure and new cell types can then be formed.

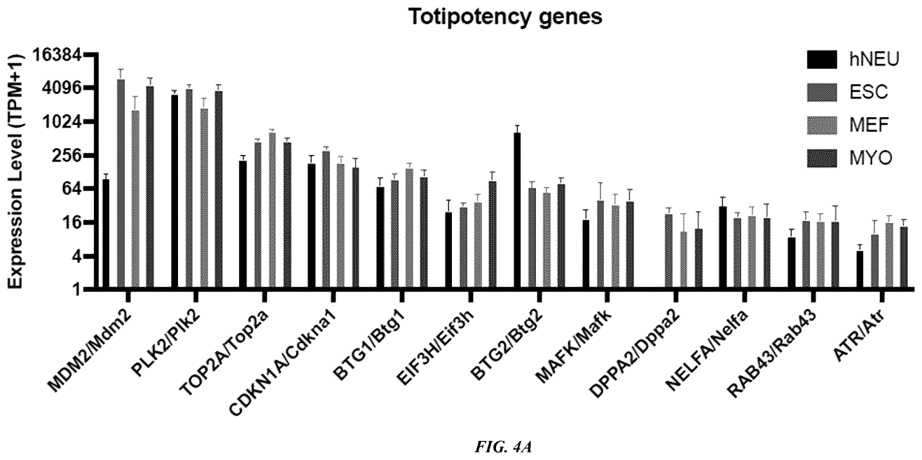

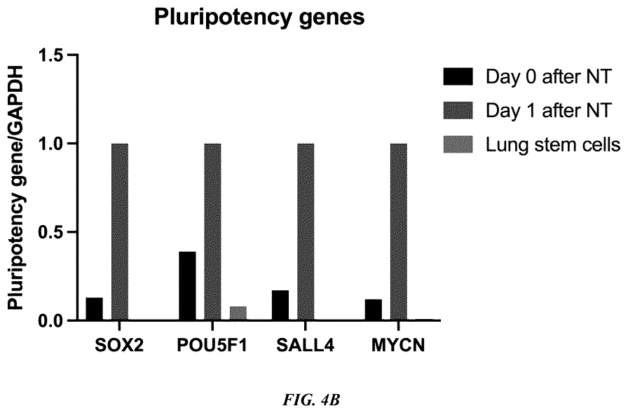

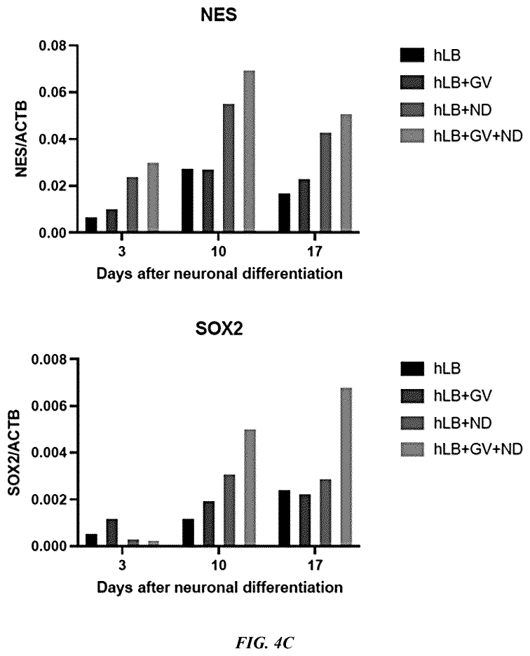

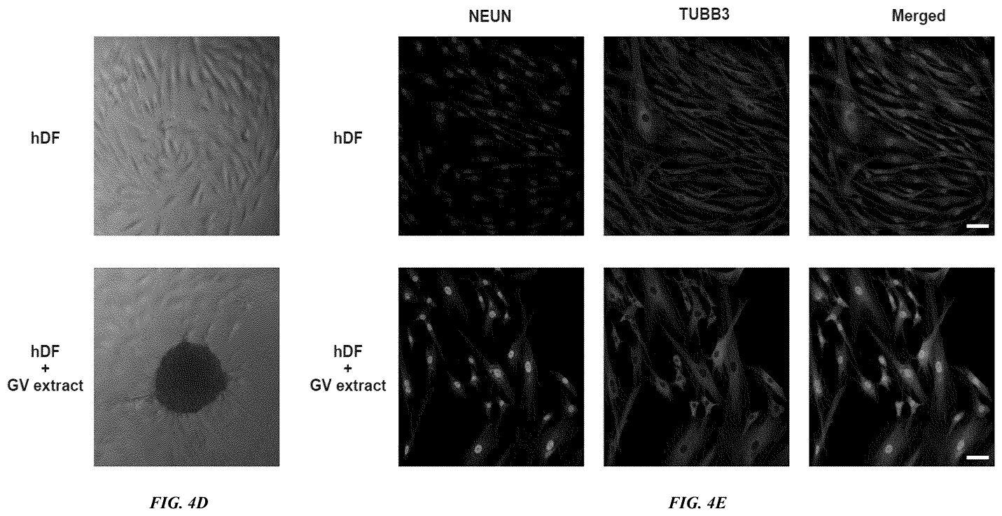

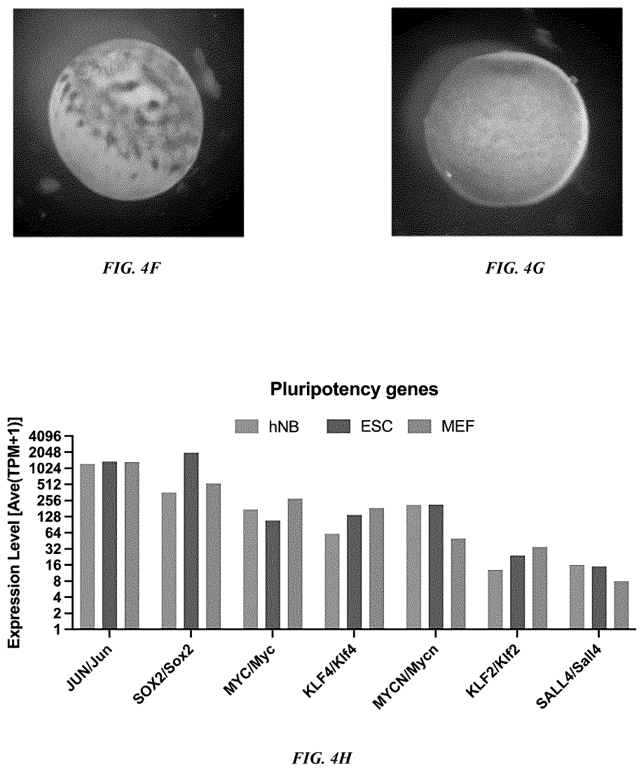

To make our GV-extract procedure applicable to human cells, we would like to be reassured that the activation of mouse and human pluripotency genes during transcriptional reprogramming is comparable. We have demonstrated that pluripotency genes are activated by Xenopus meiotic oocytes in both mouse cells (ESC and MEF) and human neuroblastoma cells (hNB) to similar levels after nuclear transfer (FIG. 4A). The activation of pluripotency genes and completion of transcriptional reprogramming in both mouse and human cells allow us to test human lung stem cells, which are directly derived from human embryos. Pluripotency genes were activated successfully for human lung stem cells via nuclear transfer to oocytes (FIG. 4B) and these may be relevant to the alleviation of human respiratory malfunction.

We therefore developed our procedure to apply our oocyte GV extracts to cultured human cells and show the activation of pluripotency genes in human blood lymphoblasts (hLB). We then tested the switch of cell types by asking whether hLBs adopt the gene expression of neural cells after 3, 10 or 17 days of culture in neuronal differentiation media (FIG. 4C). This is indeed the case, as seen in FIG. 4C where hLBs are seen to express the neuronal marker genes NES and SOX2 after being treated with GV extracts and neuron differentiation medium. This change in expression is what we look for if oocyte reprogrammed cells are to have therapeutic value.

However, the change in gene expression does not enable the suspended hLBs to be transformed into morphologically attached neuron cells. We therefore used another cell type, human dermal fibroblasts (hDF) to develop our methods. Following the same reprogramming procedures by

GV extracts, reprogrammed hDFs successfully form embryoid bodies after 4 days in culture and maintain the function as embryonic stem cells for the following days (FIG. 4D). We then treated the hDFs with neural differentiation factors for three weeks. GV-reprogrammed hDFs differentiated into neuron-like cells morphologically and expressed two neuron gene markers, NEUN and TUBB3 (FIG. 4E).

It is remarkable that Xenopus oocyte GV extracts can activate gene expression in mammalian nuclei, even though mammalian nuclei injected to eggs (FIG. 4F, in mitotic metaphase) undergo very abnormal cell division and do not permit normal development. The best explanation for this is the oocyte (FIG. 4G, in meiotic prophase) completely lacks the components of eggs that induce cell division, but already contain those components that convert oocyte chromatin into a transcriptionally active state. This active chromatin state permits gene expression but does not cause chromosome damage as induced cell division does. This is an amazing switch in activity of oocytes as they go from meiotic to mitotic activity and this is normally brought about by progesterone hormonal activity in the course of about 24 hours.

We conclude that the incubation of human cells in Xenopus oocyte GV extracts mimics the reprogramming process via nuclear transfer into GV oocytes. Transcriptionally and functionally, GV-reprogrammed human cells express pluripotency genes and gain the function of embryonic stem cells. The addition of differentiation factors allows the GV-reprogrammed human cells to gain new functions that are distinct from original cell types.

In summary, the examples show that Xenopus meiotic oocytes and GV extracts can be used to reprogram mouse nuclei towards totipotency and investigate the benefits of utilizing Xenopus meiotic oocytes to reprogram cells. Different from iPSC approach and nuclear transfer to mature eggs, nuclear transfer to meiotic oocytes does not require pioneer transcription factors or DNA synthesis/cell division but uses maternal factors to increase the accessibility of chromatin to facilitate transcriptional reprogramming of hundreds of transplanted nuclei within hours.

We first compare the results of nuclear transfer to oocytes as described here with nuclear transfer to eggs. Are the kinds of genes activated the same by these procedures? There is certainly a large difference in this respect. We find that nearly 40% of the mouse genes tested here underwent a gene expression change in oocyte nuclear transfers, in most cases towards a more totipotent level of gene expression. Some previous work has been done on the difference between nuclear reprogramming by oocytes and eggs. Alberio et al (2005) show the remodelling of nuclear lamina is similar in both oocyte and egg reprogramming; however, the transcriptional activity is different27. A major difference between oocyte nuclear transfers and iPSC approach concerns the timing of the changes induced. Most of the changes seen in oocyte nuclear transfers take place within 24 hours, and there is very little change in 48 compared to 24 hours. The rapidity of changes is similar for all of the donor cell types used here. Oocytes show fast and efficient reprogramming competence to induce a totipotency-like state, compared to iPSC methods²⁸.

We now ask whether the components of oocytes needed for reprogramming are the same as those needed for egg nuclear transfers and for iPSC approach. We think mostly not. Most of the molecules needed for egg nuclear transfers are not yet identified but are likely to do with DNA synthesis and chromatin dispersion. There is no DNA synthesis in oocytes, but the components needed for chromatin structural changes could be similar in oocytes and eggs. These could include nucleoplasmin, DNA demethylases, etc. Lastly, we ask whether the mechanisms used for reprogramming are similar for oocytes and the other routes. We have shown here that DNA synthesis and cell selection are not required in oocytes but very well may be important in iPSC method and nuclear transfer to eggs. Other reprogramming components of eggs, including Gli129 and Brg124 are not known to be required for reprogramming by oocytes. Previously, Xenopus Wave1 was shown to be essential for nuclear reprogramming in oocytes³⁰. Our future work will aim to identify other oocyte reprogramming components, using the antibody procedure of Clift et al (2017)³¹.

The main conclusion from the present nuclear transfer to oocyte experiments and GV extract treatment is that the most effective somatic reprogramming factors are present in the germinal vesicles of meiotic oocytes, and do not require cell division or cell selection. The use of maternal factors of oocytes are different from the use of Yamanaka factors to reprogram cells. Potentially, oocyte reprogramming factors can combine with other factors to enhance the somatic cell nuclear reprogramming and execute the conversion of cell types.

### Example 5 GV Modification

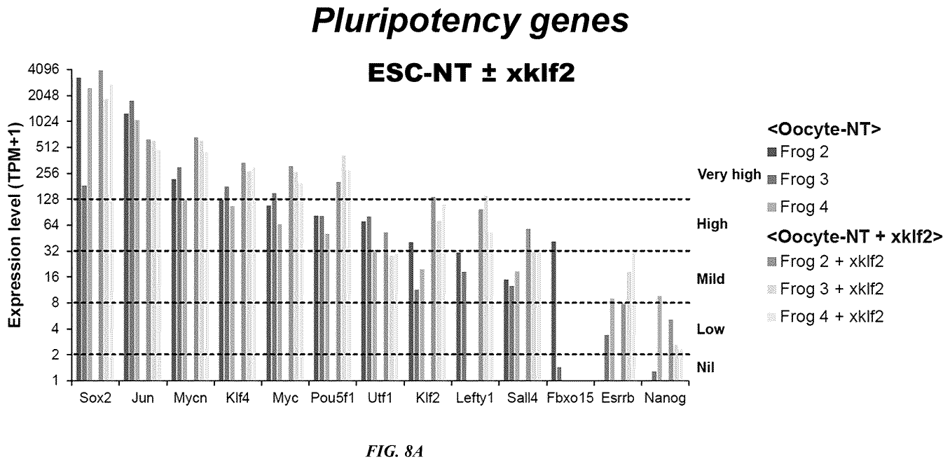

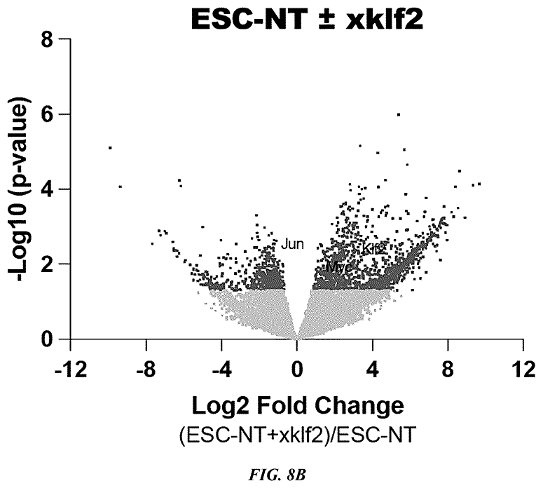

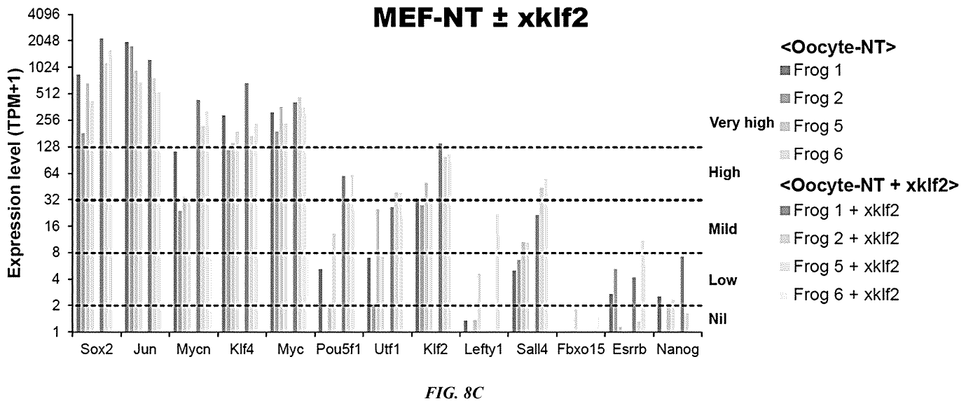

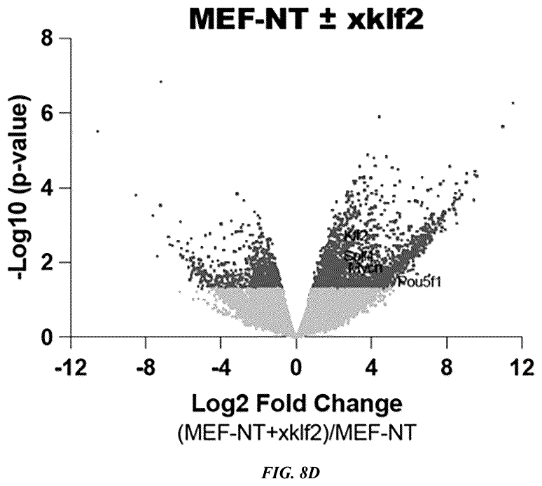

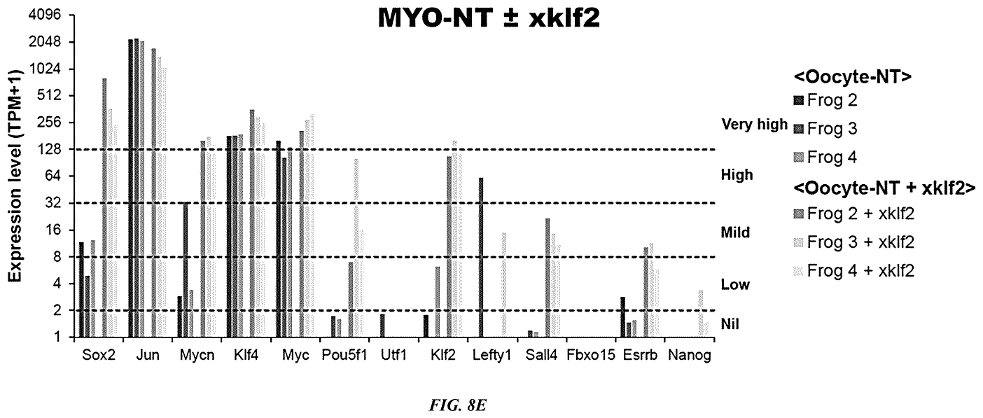

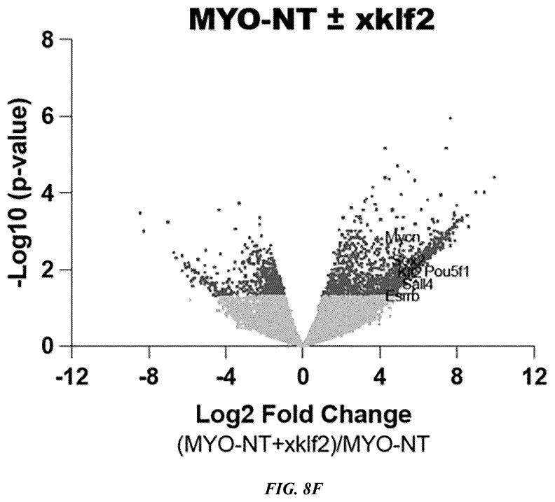

9.2 ug xklf2-HA mRNA (SEQ ID NO: 1) was injected into the cytosol of oocytes 24 hours before nuclear transfer of mammalian nuclei into Xenopus oocytes (FIG. 8). The overexpression of xklf2-HA upregulates 1000-2000 genes in mouse cells, ESC, MEF, and MYO. Within these genes, reprogramming resistant genes, Pou5f1 in MEF, and Sox2, Mycn, Pou5f1, Klf2, Sall4, and Esrrb in MYO were activated by xklf2-HA overexpression.

### Materials and Methods

Isolation of oocytes from female frogs Oocyte material could probably be obtained from any vertebrate ovary. Xenopus is particularly suitable, because a normal female, weighing 170 grams has about 5,000 oocytes in its ovary, which weighs 30 grams. The most benign and painless procedure for obtaining oocytes from Xenopus is to subcutaneously inject female frog with 3 grams of Ethyl 3-aminobenzoate methanesulfonate salt (otherwise known as MS 222). This is best made up in a one-half ml of water. The injected frog is kept on ice on its back for 15 mins after which it should be fully anaesthetized and it cannot turn itself the right way round. The ovary material is removed from the frog and it is torn up into strings so each oocyte is exposed to the medium. The necessary amount of ovarian material, usually about 3 ml of ovarian tissue without medium, is added to material called liberase (250 ul, 28U/ml in H2O, Roche, 5401020001) and this is made up in 12.5 ml MBS medium and placed on a slow rocker so that the ovarian material is fully exposed to the liberase. This is left on the rocker for 2 hours at room temperature, after which nearly all oocytes should be free of ovarian material, though each is covered with a single layer of follicle cells which helps protect the oocyte for injection. Then the liberase was washed away from the oocytes with 1×MBS and the stage V/VI oocytes³² with diameter range of 1 to 1.2 mm were selected for the following experiments. The selected oocytes were placed in a petri dish in 1×MBS at 16° C. and the follicular cell layer will detach in an overnight incubation. The oocytes obtained are then put into MBS medium, containing 0.1% bovine serum albumin and antibiotics. The oocytes can be kept at 16° C. for several days.

### Cell Culture

Mouse embryonic stem cells were cultivated in G-MEM BHK-21 (Gibco, 21710-025), 20% fetal bovine serum (Gibco, 10439-024), 1000U/ml leukemia inhibitory factor (Chemicon, ESG1107), 0.1 mM non-essential amino acids, 0.1 mM β-mercaptoethanol and 10 mM sodium pyruvate in a gelatin coated flask. A mouse embryonic fibroblast cell line³³ and a mouse myoblast cell line, C2C12, were cultured in DMEM (Sigma, D5671) with 10% fetal bovine serum (ThermoFisher Scientific, 26140079). Human lung stem cells were derived from fetal lung epithelial tips and grown as long-term self-renewing organoids³⁴. Human lymphoblast cell line K-562 (Merck, 89121407-1VL) were cultivated in IMDM (ThermoFisher Scientific, 31980030) with 10% fetal bovine serum. Human adult dermal fibroblasts (ThermoFisher Scientific, C0135C) were cultivated in medium 106 (ThermoFisher Scientific, M106500) with low serum growth supplement kit (ThermoFisher Scientific, S003K).

### Cell Permeabilization

Cells were cultured to sub-confluence, washed twice with PBS and detached with trypsin for 5 mins at 37° C. Trypsin was neutralized by culture medium with BSA and centrifuged at 500 rpm for four minutes. The supernatant was discarded, and the cells were resuspended in PBS. Cells were centrifuged at 2000 rpm for one minute and PBS was replaced with SuNaSp solution (250 mM Sucrose, 75 mM NaCl, 0.5 mM Spermidine, 0.15 mM Spermine). Cells were centrifuged at 2000 rpm for one minute and the supernatant was discarded. 20 ul streptolysin O (SLO, 20,000 units/ml in PBS, containing 0.01% BSA and 5 mM DTT, Sigma-Aldrich, S5265) was added plus 100 ul SuNaSp solution for 3-6×106 cells. Cells were then resuspended by pipetting and permeablized at 37° C. in a waterbath for one minute. Cells were incubated on ice and some cells were taken to check permeablization efficiency (95˜99%) under a microscope by trypan blue staining. SLO reaction was stopped by adding SuNaSp BSA. Cells were centrifuged at 2000 rpm for one minute, the supernatant was discarded and then resuspended with SuNaSp BSA solution for cell concentration at ˜500 nuclei/10 nl. Cells in SuNaSp BSA solution were then aliquotted, snap-frozen on dry ice and stored in a −70° C. freezer.

### Cytoplasmic Injection

For xklf2 overexpression, xklf2 mRNA (SEQ ID NO: 1) was in vitro synthesized from linearized pCS2-xklf2-HA plasmid by MEGAscript SP6 (Invitrogen, AM1330). xklf2 mRNA, 9.2 ng, was injected into cytoplasm of oocytes 24 hours before nuclear transfer. The injection of BrUTP into cytoplasm was two hours after nuclear transfer and 5 nl BrUTP solution (100 mM in H2O, Sigma, B7166) was injected into the cytoplasm of each oocyte.

### Nuclear Transfer

Permeabilized cells mixed with plasmid DNA encodes cytoplasmic membrane GFP35. Inject 9.2 nl cell suspension (˜500 cells) with 5 pg plasmid of cytoplasmic membrane GFP into germinal vesicle of each oocyte. The permeabilized cells were injected into the GV of oocytes via Drummond Nanoinjector. Select GFP positive oocytes for oocytes collected beyond 24 hours of nuclear transfer; within 24 hours, no GFP selection. Snap-freeze samples on dry ice and store samples in −70° C. freezer.

### Confocal Microscopy and Analysis

Immunofluorescence of HP1α and Lamin-A/C is referred to Nguyen T et al., 201936. After nuclear transfer, oocytes were fixed in low FG fixative and then post-fix with MeOH/EGTA. Rehydrate the oocytes in a sequence of 25%, 50%, 75% and 100% TBS/MeOH and hemisect oocytes. Bleach oocytes with bleach solution (1% H2O2, 5% formamide, 150 mM NaCl, 16 mM sodium citrate, pH7.0 adjusted by NaOH). Stain the transplanted nuclei with anti-HP1α antibody (Alexa Fluor 647, Abcam, ab 198391) or anti-Lamin-A/C antibody (Alexa Fluor 680, Santa Cruz, sc-376248) and stain the chromatin by DNA dye, TO-PRO-1 (Thermo Fisher, T3602). Clear oocytes by Murray's clearing solution for microscopic imaging by Zeiss LSM880 Confocal Microscope. Chromatin density was quantified using a custom script for Fiji which applies a multi-level Otsu threshold with the histogram for calculation limited to values greater than or equal to the threshold from the previous iteration (https://github.com/gurdon-institute/DNA_Density/blob/main/Wen-Butler_Chromatin_Density.py). Areas of nuclei were measured by segmenting the Huang thresholded masks of TO-PRO-1 images using a recursive algorithm which watersheds sub-regions using decreasing distance map tolerance values, and extracts objects that are small enough to be considered individual nuclei at each stage (https://github.com/gurdon-institute/DNA_Density/blob/main/Wen-Butler_Recursive_Watershed.py). An area overlaying chromatin of injected nuclei is marked, and each pixel in that area is scored for signals from the TO-PRO-1 labelled chromatin. This DNA dye TO-PRO-1 was used to classify the chromatin into three density levels that are loose, middle, or dense. The heterochromatin is indicated by an antibody against heterochromatin protein 1 alpha, whose signal was scored for heterochromatin area. Single molecule RNA fluorescence in situ hybridization nuclear transfer, oocytes were fixed in 1% MEMFA and then immersed in 100% MeOH at −20° C. for at least 48 hours. Rehydrate oocytes with a series of TBST/MeOH (25%, 50%, 75% and 100%), two times for each solution for at least 30 mins. Hemisect the oocytes and bleach oocytes with 1% H2O2, 5% formamide and 1×SSC for 16 hours. Wash oocytes with the TBST for 30 mins twice and replace with Stellaris Wash Buffer A (LGC Biosearch Technologies) for 30 mins at RT. Remove Stellaris Buffer A and immerse oocytes in Stellaris Hybridization Buffer (LGC Biosearch Technologies) and allow oocytes to settle for 5 mins. Remove Hybridization Buffer and immerse oocytes in 125 nM probe working solution of Sox2 (Stellaris RNA FISH Probes of Sox2, LGC Biosearch Technologies) at 37° C. in the dark for 16 hours. Remove the probe working solution and wash with Hybridization Buffer. Stain chromatin by 5 μM TO-PRO-1, diluted by Wash Buffer A. Wash with TBST for 15 mins twice. Postfix by 1% formaldehyde/TBST at RT for 1 hour. Wash with TBST for 30 mins twice and dehydrate oocytes with 100% MeOH for 16 hours. Remove MeOH and add Murray's clearing solution. Allow oocytes to set at the bottom of tubes and image oocytes by Zeiss LSM880 Confocal Microscope.

### RNA Extraction

Collect Oocyte-NT samples as groups of ten for RNA extraction. Qiagen RNeasy Mini Kit (QIAGEN, 74104) is used for RNA extraction and procedures are modified for our purpose. Briefly, lyse Oocyte-NT samples with 900 ul RLT buffer and votex for 4 minutes at 4° C. Add 900 ul 70% ethanol and transfer mixture to RNeasy spin column. Centrifuge at 10,000 rpm for 30 seconds and discard flow-through. Add 350 ul RW 1 buffer and centrifuge at 10,000 rpm for 30 seconds. Discard flow-through and add 80 ul DNase I incubation mix. Incubate at room temperature for 15 minutes, add 350 ul RW 1 buffer and centrifuge at 10,000 rpm for 30 seconds. Discard flow-through and add 500 ul RPE buffer and centrifuge at 10,000 rpm for 2 minutes. Place RNeasy spin column in new 2 ml collection tubes and centrifuge at full speed for 1 minute. Place RNeasy spin column in new 1.5 ml eppendorf and add 50 ul RNase-free H2O. Centrifuge at 10,000 rpm for 1 minute and measure RNA concentration by Nanodrop. Snap-freeze RNA extracts on dry ice and store RNA extract in −70° C. freezer.

### Reverse Transcription and QPCR

SuperScript III Reverse Transcriptase Kit (ThermoFisher, 18080) is used for reverse transcription. SYBR Green JumpStart Taq Ready Mix (Sigma-Aldrich, S9939) is used for QPCR. Primers for pluripotency genes are listed in Halley-Stott's paper35. 50 ng cDNA per well of a QPCR plate is used and Gapdh is used for normalization.

### Immunoprecipitation of BrUTP Incorporated RNA

The protocol is adapted from a published protocol 19. Wash the anti-BrUTP conjugated agarose beads (Santa Cruz Biotechnology, sc-32323 AC) twice with Buffer I (0.5×SSPE with 0.05% Tween 20 and 0.1% polyvinylpyrrolidone) and block beads with Blocking buffer (Buffer I with 1 mg/ml RNase free BSA) at 4° C. for 1.5 hours. Centrifuge beads solution at 3000 rpm for 3.5 minutes at 4° C. and remove supernatant. Use 25-50 ug RNA extract per sample and add 2.5 ul SUPERase·In RNase Inhibitor (ThermoFisher, AM2696) into each sample. Heat RNA extract at 65° C. for 5 minutes, incubate on ice for at least 1 minute and spin down. Immunoprecipitate RNA with 200 ul RIP buffer (anti-BrUTP beads in 0.5×SSPE with 0.05% Tween 20) overnight at 4° C. Wash RNA bead mixture with Low salt buffer (0.2×SSPE with 0.05% Tween 20), with High salt buffer (0.5×SSPE with 0.05% Tween 20 and 150 mM NaCl) twice and TET buffer (10 mM Tris, 1 mM EDTA, pH 8 with 0.05% Tween 20). Elute immunoprecipitated RNA with Elution buffer (5 mM Tris, pH 7.5 with 300 mM NaCl, 1 mM EDTA, 0.1% SDS, 20 mM dithiothreitol) by incubating at room temperature for 1 minute. Centrifuge at 3000 rpm for 4 minutes and collect supernatant. Repeat the elution steps 4 times. Extract eluted RNA by phenol/chloroform extraction and ethanol precipitation. Clean up RNA extract with

### Qiagen RNeasy Plus Micro Kit.

Library preparation and sequencing of RNA-seq Ovation Single Cell RNA-seq System (NuGEN, Part No 0342) is used to prepare RNA-seq libraries from newly-synthesized RNA. 10 ng of newly-synthesized RNA was used for each sample preparation. Follow the steps provided by manufacturers. cDNA reverse transcribed from newly-synthesized RNA is then obtained and amplified as RNA-seq libraries. RNA-seq libraries were validated by Agilent 2200 TapeStation and sequenced on Illumina HiSeq 2000 and 4000 for SE50.

### Filtering and Mapping of Sequencing Data

Fasta files from Mus musculus (mm10) and Xenopus laevis (xla9.1) have been concatenated one after the other in order to create a hybrid large mouse-Xenopus genome. To distinguish the X. laevis chromosomes in the fasta file, they have been renames as “xla_chr” instead of just “chr”. Similarly, the gtf files containing the annotation of all transcripts from Mouse (mm10) and of all primary transcript from X. laevis (xla9.1) have been concatenated. FastQ files were processed with cutadatp (version 1.9.1, options −q 10 −O 3) for adapter trimming. Filtered reads were then aligned to the hybrid mouse-Xenopus genome with tophat (version v2.1.1). Transcripts were assigned to gene and counted using htseq-count (HTSeq-0.5.4p3).

### In Vitro Reprogramming by GV Extracts

Oocytes are released from ovaries of Xenopus laevis (see above, Isolation of oocytes from female frogs). Dissect GVs manually in the mineral oil and collect the GVs in a tube. Snap freeze the GVs on dry ice and store in −70° C. freezer. Add calcium-free IMDM medium (United States Biological, 18750-08) and centrifuge at 16,100g for 10 mins at 4° C. Take supernatant and place GV extract on ice, or at −20° C., for later use. Human lymphoblasts K-562 and human adult dermal fibroblasts were permeabilized by SLO in PBS (see above, Cell permeabilization). Permeabilized cells were then cultured in calcium-free IMDM medium with GV extract for 6 hours and added culture medium with CaCl2 (final 2 mM) for resealing membrane.

### Neuronal Differentiation

The neuronal differentiation protocol was modified from a previously published paper28 (Giulitti et al., 2019). GV-extract reprogrammed cells were seeded on Matrigel-coated plates at a density of 530 cells/mm2 and cultured in mTeSR1 Plus medium (STEMCELL Technology, 100-0276) for six days. From day 0 to 2, cells were cultured in neural medium (N2B27, 1% NEAA, 200 ng/ml L-ascorbic acid) added with 20 ng/ml bFGF (STEMCELL Technology, 78003.1) and 0.1 μM LDN 193189 (STEMCELL Technology, 72147). The cells were then cultured in neural medium supplemented with 0.1 μM LDN 193189 and 10 μM SB431542 (STEMCELL Technology, 72234) at day 3. The following 6 days, the same medium used at day 3 was supplemented with 1 μM all-trans retinoic acid (Merck, R2625) and 1 μM SAG (Merck, 566661). On day 10 to 15, the cells were cultured in neural medium supplemented with 5 μM DAPT (Merck, D5942), 4 μM SU-5402 (Merck, SML0443), 1 μM all-trans retinoic acid and 1 μM SAG. On day 16, cells were seeded on glass-bottom chamber slide and cultured in maturation medium, which contains neural medium supplemented with 20 ng/ml BDNF, 10 ng/ml GDNF, 10 ng/ml CNTF (PeproTech, 450-02, 450-10, 450-13) and 10 μM ROCK inhibitor (STEMCELL Technology, 72302). The cells were cultured in maturation medium up to day 22 and fixed for immunostaining. All the culture media were changed daily.

### REFERENCES

1. Takahashi, K. & Yamanaka, S. Induction of pluripotent stem cells from mouse embryonic and adult fibroblast cultures by defined factors. Cell 126, 663-676 (2006).
2. Gurdon, J. B., Elsdale, T. R. & Fischberg, M. Sexually mature individuals of Xenopus laevis from the transplantation of single somatic nuclei. Nature 182, 64-65 (1958).
3. Soufi, A. et al. Pioneer transcription factors target partial DNA motifs on nucleosomes to initiate reprogramming. Cell 161, 555-568 (2015).
4. Soufi, A., Donahue, G. & Zaret, K. S. Facilitators and impediments of the pluripotency reprogramming factors' initial engagement with the genome. Cell 151, 994-1004 (2012).
5. Buganim, Y. et al. Single-cell expression analyses during cellular reprogramming reveal an early stochastic and a late hierarchic phase. Cell 150, 1209-1222 (2012).
6. Lavagnolli, T. et al. Initiation and maintenance of pluripotency gene expression in the absence of cohesin. Genes & development 29, 23-38 (2015).
7. Jullien, J. et al. Hierarchical molecular events driven by oocyte-specific factors lead to rapid and extensive reprogramming. Molecular cell 55, 524-536 (2014).
8. Polo, J. M. et al. A molecular roadmap of reprogramming somatic cells into iPS cells. Cell 151, 1617-1632 (2012).
9. Pasque, V., Miyamoto, K. & Gurdon, J. B. Efficiencies and mechanisms of nuclear reprogramming. Cold Spring Harbor symposia on quantitative biology 75, 189-200 (2010).
10. Wakayama, T., Tateno, H., Mombaerts, P. & Yanagimachi, R. Nuclear transfer into mouse zygotes. Nat Genet 24, 108-109 (2000).
11. Kang, E. et al. Nuclear reprogramming by interphase cytoplasm of two-cell mouse embryos. Nature 509, 101-104 (2014).
12. Do, J. T. & Scholer, H. R. Nuclei of embryonic stem cells reprogram somatic cells. Stem cells 22, 941-949 (2004).
13. Han, D. W. et al. Pluripotential reprogramming of the somatic genome in hybrid cells occurs with the first cell cycle. Stem cells 26, 445-454 (2008).
14. Ganier, O. et al. Synergic reprogramming of mammalian cells by combined exposure to mitotic Xenopus egg extracts and transcription factors. Proceedings of the National Academy of Sciences of the United States of America 108, 17331-17336 (2011).
15. Gurdon, J. B. Injected nuclei in frog oocytes: fate, enlargement, and chromatin dispersal. J Embryol Exp Morphol 36, 523-540 (1976).
16. Machida, S. et al. Structural Basis of Heterochromatin Formation by Human HP1. Molecular cell 69, 385-397 e388 (2018).
17. Bannister, A. J. et al. Selective recognition of methylated lysine 9 on histone H3 by the HP1 chromo domain. Nature 410, 120-124 (2001).
18. Matoba, S. et al. Embryonic development following somatic cell nuclear transfer impeded by persisting histone methylation. Cell 159, 884-895 (2014).
19. Jullien, J. et al. Gene Resistance to Transcriptional Reprogramming following Nuclear Transfer Is Directly Mediated by Multiple Chromatin-Repressive Pathways. Molecular cell 65, 873-884 e878 (2017).
20. Harr, J. C. et al. Directed targeting of chromatin to the nuclear lamina is mediated by chromatin state and A-type lamins. The Journal of cell biology 208, 33-52 (2015).
21. van Steensel, B. & Belmont, A. S. Lamina-Associated Domains: Links with Chromosome Architecture, Heterochromatin, and Gene Repression. Cell 169, 780-791 (2017).
22. Liu, K., Song, Y., Yu, H. & Zhao, T. Understanding the roadmaps to induced pluripotency. Cell Death Dis 5, e1232 (2014).
23. King, T. J. & Briggs, R. Changes in the Nuclei of Differentiating Gastrula Cells, as Demonstrated by Nuclear Transplantation. Proceedings of the National Academy of Sciences of the United States of America 41, 321-325 (1955).
24. Hansis, C., Barreto, G., Maltry, N. & Niehrs, C. Nuclear reprogramming of human somatic cells by xenopus egg extract requires BRG1. Current biology: CB 14, 1475-1480 (2004).
25. Guan, J. et al. Chemical reprogramming of human somatic cells to pluripotent stem cells. Nature 605, 325-331 (2022).
26. Gill, D. et al. Multi-omic rejuvenation of human cells by maturation phase transient reprogramming. Elife 11 (2022).
27. Alberio, R., Johnson, A. D., Stick, R. & Campbell, K. H. Differential nuclear remodeling of mammalian somatic cells by Xenopus laevis oocyte and egg cytoplasm. Experimental cell research 307, 131-141 (2005).
28. Giulitti, S. et al. Direct generation of human naive induced pluripotent stem cells from somatic cells in microfluidics. Nature cell biology 21, 275-286 (2019).
29. Maekawa, M. et al. Direct reprogramming of somatic cells is promoted by maternal transcription factor Glis1. Nature 474, 225-229 (2011).
30. Miyamoto, K. et al. Nuclear Wave1 is required for reprogramming transcription in oocytes and for normal development. Science 341, 1002-1005 (2013).
31. Clift, D. et al. A Method for the Acute and Rapid Degradation of Endogenous Proteins. Cell 171, 1692-1706 e1618 (2017).
32. Dumont, J. N. Oogenesis in Xenopus laevis (Daudin). I. Stages of oocyte development in laboratory maintained animals. J Morphol 136, 153-179 (1972).
33. Pasque, V., Gillich, A., Garrett, N. & Gurdon, J. B. Histone variant macroH2A confers resistance to nuclear reprogramming. The EMBO journal 30, 2373-2387 (2011).
34. Nikolic, M. Z. et al. Human embryonic lung epithelial tips are multipotent progenitors that can be expanded in vitro as long-term self-renewing organoids. Elife 6 (2017).
35. Halley-Stott, R. P. et al. Mammalian nuclear transplantation to Germinal Vesicle stage Xenopus oocytes—a method for quantitative transcriptional reprogramming. Methods 51, 56-65 (2010).
36. Nguyen, T., Mitchison, T. J. & Wuhr, M. Immunofluorescence of Microtubule Assemblies in Amphibian Oocytes and Early Embryos. Methods Mol Biol 1920, 17-32 (2019).
37. Bui, H-T., et al. Epigenetic reprogramming in somatic cells induced by extract from germinal vesicle stage pig oocytes. Development. 139, 4330-4340 (2012).
38. Hammelman, J., et al. Ranking reprogramming factors for cell differentiation. Nature methods. 19, 812-822 (2022).

1. A method of nuclear reprogramming somatic cells to alter the cell type, the method comprising:
preparing a germinal vesicle (GV) extract;
permeabilising somatic cells;
incubating the somatic cells with the GV extract to alter the cell type; and
resealing somatic cell membranes,
wherein said GV extract does not comprise oocyte cytoplasm.

2. The method according to claim 1, wherein the altering the cell type comprises reprogramming the somatic cell to a progenitor cell or stem cell.

3. The method according to claim 2, further comprising differentiating the progenitor cell or the stem cell to alter the cell type.

4. The method according to claim 3, wherein differentiating the progenitor cell or the stem cell produces a somatic cell or a progenitor thereof of a different lineage.

5. The method according to claim 3, wherein differentiating comprises incubating the progenitor cell or the stem cell with differentiation medium comprising one or more differentiation factors.

6. The method according to claim 1, wherein the GV extract comprises a reprogramming factor.

7. The method according to claim 6, wherein the reprogramming factor is a factor which changes chromatin accessibility.

8. The method according to claim 7, wherein the reprogramming factor is a DNA modifying enzyme, a histone variant, a histone-modifying enzyme, a chromatin remodeler, a chromatin modifier, or a transcription factor.

9. The method according to claim 8, wherein the DNA modifying enzyme is AID or Mbd3, the histone-modifying enzyme is kdm4 or kdm6, the chromatin remodeler is Brg1, and/or the transcription factor is Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, or a homologue thereof.

10. The method according to claim 1, wherein the GV extract is derived from a meiotic oocyte.

11. The method according to claim 1, wherein the GV extract is from a Xenopus, a mammal, or a ray-finned fish.

12. The method according to claim 1, wherein the somatic cells are mammalian cells.

13. The method according to claim 1, further comprising incubating the somatic cells and the GV extract with an exogenous reprogramming factor.

14. The method according to claim 1, wherein the somatic cells and the GV extract are incubated for any one of about 1, 2, 3, 4, 5, 6, 7, 8, 9, or 10 days.

15. The method according to claim 1, wherein the method further comprises a step of modifying the GV extract to express or overexpress a gene encoding a protein selected from Gli1, FoxA, Gata4, Ascl1, Brn2, Myt1l, PU.1, xklf2, xsox2, xpou60, mouse mSox2, mFoxa1, hOCT4, hKLF4, Oct3/4, Sox2, Klf4, MyoD, Gata2/3, Foxa1, Hnf4a, Hnf1a, Pax4, Pdx1, and homologues thereof.

16. The method according to claim 15, wherein the step of modifying the GV extract comprises injecting the cytoplasm with mRNA or incubating the oocyte with a rare-cutting endonuclease comprising a TALEN, a ZFN or a CRISPR/Cas9 and guide RNA, optionally, wherein the mRNA encodes a protein selected from Gli1, FoxA, Gata4, Ascl1, Brn2, Myt1l, PU.1, xklf2, xsox2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, and homologues thereof, or the guide RNA comprises a gene encoding Gli1, FoxA, Gata4, Ascl1, Brn2, Myt1l, PU.1, xklf2, xsox2, xklf2, xpou60, mouse mSox2, mFoxa1, human hOCT4, hKLF4, Oct3/4, Sox2, Klf4, MyoD, Gata2/3, Foxa1, Hnf4a, Hnf1a, Pax4, Pdx1, or homologues thereof.

17. A germinal vesicle (GV) extract comprising reprogramming factors, optionally, wherein the reprogramming factors are factors which change chromatin accessibility.

18. A cell derived from the method of claim 1.

19. A cell with an altered cell type derived from the method of claim 1.

20. A kit comprising a permeabilization agent, a GV extract, and one or more reprogramming factors, and optionally instructions for use.
